(19) United States
(12) Patent Application Publication
(10) Pub. No.: US 2025/0319211 A1
(43) Pub. Date: Oct. 16, 2025

(54) NEAR IR LUMINESCENCE AND OPTICALLY ADDRESSABLE QUANTUM SENSING AND MAGNETIC IMAGING WITH RADICALOID TETRATHIAFULVALENE TETRATHIOLATES

(71) Applicant: The University of Chicago, Chicago, IL (US)

(72) Inventors: John Stuart Anderson, Chicago, IL (US); Lauren McNamara, Chicago, IL (US)

(21) Appl. No.: 18/866,043

(22) PCT Filed: May 16, 2023
(86) PCT No.: PCT/US2023/022433

§ 371 Date: Nov. 14, 2024

Related U.S. Application Data
(60) Provisional application No. 63/364,865, filed on May 17, 2022.

(51) Int. Cl. A61K 49/00

(52) U.S. Cl. CPC A61K 49/0021

(57) ABSTRACT

Tetrathiafulvalene-2,3,6,7-tetrathiolate (TTFtt) bridged bimetallic complexes with radical character which are bright, air- and water-stable, persistent and exhibit excellent near-infrared photophysical properties; and methods of use of the TTFtt complexes for imaging, guidance of surgery, as qubits, and for interventional medical treatments as theranostic agents are described.

### INCORPORATION BY REFERENCE

A PCT Request Form is filed concurrently with this specification as part of the present application. Each application that the present application claims benefit of or priority to as identified in the concurrently filed PCT Request Form is incorporated by reference herein in their entireties and for all purposes.

### STATEMENT OF GOVERNMENT SUPPORT

This invention was made with government support under grant number DE-SC0019215 awarded by the U.S. Department of Energy; grant number W911NF-20-1-0091 by the Army Research Office; and grant number DMR-1420709 by the National Science Foundation. The government has certain rights in the invention.

### FIELD OF THE INVENTION

The present disclosure relates to dicationic tetrathiafulvalene-2,3,6,7-tetrathiolate (TTFtt) bridged bimetallic compounds with diradical character exhibiting excellent near-infrared properties, that are bright, air- and water-stable and persistent. They have utility for imaging, guidance of surgery, as qubits and for interventional medical treatments as theranostic agents.

### BACKGROUND OF THE INVENTION

Molecular near-infrared (NIR) dyes and lumiphores have attracted attention due to their promising applications in biological imaging and the development of lasers, detectors, and organic light-emitting diodes (OLEDs). The NIR region (700 nm-1700 nm) falls in the tissue transparent window, so NIR emitters are often implemented for in vivo, in vitro, and in intraoperative imaging applications. For these applications, the main challenges facing current NIR dye development are autofluorescence, scattering, and water overtones. Both autofluorescence and scattering are dramatically reduced in the NIR II region, but water absorptions at ˜1400 nm pose a significant obstacle for many NIR dyes which frequently have low photoluminescence quantum yields (PLQY). The physical underpinnings of emission in the NIR region makes low PLQYs a considerable challenge facing current dye candidates. PLQYs are limited by the energy gap law, which theorizes that emission efficiency is lowered for low energy transitions due to exponentially increasing nonradiative decay rates. For molecular organic NIR dyes, these transition probabilities are often dominated by C—H modes.

These limitations on PLQY make the development of efficient NIR dyes challenging. Only two NIR dyes, indocyanine green (ICG) and methylene blue (MB), have been approved by the U.S. FDA, and both emit around 700 nm-800 nm where autofluorescence and scattering can be problematic. In addition to these thiazine and cyanine dyes, donor-acceptor-donor (D-A-D) and polymethine dyes have also been explored to further red-shift emission into the NIR II region. While they are often synthetically challenging, such donor-acceptor and D-A-D systems have recently been successfully employed to synthesize the first organic NIR dyes with emission maxima ≥1200 nm; however, these dyes exhibit extremely low PLQYs (≤ 0.05%). Furthermore, the efforts to increase the water solubility of large D-A-D systems often result in dramatically decreased PLQYs in aqueous solution. For example, the first water soluble D-A-D dye, CH1055-PEG, exhibited a ˜20×PLQY decrease in water compared to that of the parent compound (CH1055) in toluene.

Polymethine dyes are also promising candidates for bright NIR II emission. IR 26 is the brightest commercially available molecular dye with an emission maximum ≥1100 nm. While this dye emits around 1130 nm in 1,2-dichloroethane, it still exhibits a lower PLQY (0.05%) due to the low emission energy.

In addition to poor PLQY values, the molecular size and complexity required to red shift emission into the NIR region poses significant synthetic and solubility challenges. The requirement for large conjugated molecular systems makes rationally designing stimuli responsive chromophores, for instance those that turn on or off in specific chemical or electrochemical environments, extremely challenging. It would be advantageous to generate a compact and hence modular NIR II emitting moiety with high PLQY that could be tuned for some of these responsive applications.

Tetrathiafulvalenes (TTFs) can act as molecular redox switches, as they are reversibly oxidizable to dications or stable radical cations without decomposition or side reactions. Accordingly, TTFs may advantageously be employed as building blocks for NIR emitting redox switchable molecules.

The background description provided herein is for the purposes of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY OF THE INVENTION

Dicationic tetrathiafulvalene-2,3,6,7-tetrathiolate bridged bimetallic compounds with diradical character are described which have extremely bright near IR emission, are photostable, persistent in ambient conditions and aqueous mixtures and are redox-switchable for several cycles. The unique properties of these compounds are advantageous for tunable, responsive, and bright NIR dyes which can be used in various applications including NIR sensing, quantum sensing, and NIR stimulated dynamic nuclear polarization.

Accordingly, in a first aspect, the present disclosure encompasses a method of imaging a target. In some embodiments, the method includes administering a detectably effective amount of a tetrathiafulvalene-2,3,6,7 tetrathiolate bridged bimetallic complex to a target in vivo or in vitro; illuminating the target with a light source emitting light of at least one wavelength or wavelength band causing the tetrathiafulvalene-2,3,6,7 tetrathiolate bridged bimetallic complex to luminesce; and detecting luminescence with an image detector.

In some embodiments, the tetrathiafulvalene-2,3,6,7 tetrathiolate bridged bimetallic complex is a compound of Formula (I):

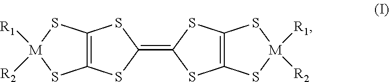

wherein M is a metal; R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring, and salts thereof.

In some embodiments, the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

In some embodiments, the tetrathiafulvalene-2,3,6,7 tetrathiolate bridged bimetallic complex is a salt having (i) an oxidation product of a compound of Formula (I), wherein the oxidation product comprises a dication diradical, dication or cation of the compound, and (ii) at least one anion.

In some embodiments, the tetrathiafulvalene-2,3,6,7 tetrathiolate bridged bimetallic complex is a salt having (i) an oxidation product of a compound of Formula (I), wherein M is selected from the group consisting of Pt and Pd; R₁ and R₂ are independently selected from the group consisting of: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring; and wherein the oxidation product comprises a dication diradical, dication or cation of the compound, and (ii) at least one anion selected from the group consisting of borates, phosphates, triflates and antimonates; and wherein the at least one anion is optionally covalently bound to at least one capping ligand.

In some embodiments the tetrathiafulvalene-2,3,6,7 tetrathiolate bridged bimetallic complex [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂. [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, or [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

In some embodiments, the target is an organ, a tissue, a muscle, a ligament, a tumor, a surgical site, a sub-cutaneous site, a cell, a neuron, lymphocyte or blood.

In a second aspect, the present disclosure encompasses a method of treating a tumor in a patient. In some embodiments, the method includes administering a detectably and therapeutically effective amount of a conjugate composed of a tetrathiafulvalene-2,3,6,7 tetrathiolate bridged bimetallic complex, and an antineoplastic agent to a site of a tumor;

illuminating the site with a light source emitting light of at least one wavelength or wavelength band causing the tetrathiafulvalene-2,3,6,7 tetrathiolate bridged bimetallic complex to luminesce;

detecting luminescence with an image detector; and treating the tumor with the antineoplastic agent.

In some embodiments, the tetrathiafulvalene-2,3,6,7 tetrathiolate bridged bimetallic complex is a compound of Formula (I):

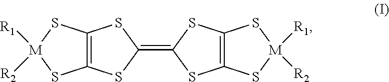

wherein M is a metal; R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring, and salts thereof.

In some embodiments, the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

In some embodiments, the tetrathiafulvalene-2,3,6,7 tetrathiolate bridged bimetallic complex is a salt having (i) an oxidation product of a compound of Formula (I), wherein the oxidation product comprises a dication diradical, dication or cation of the compound, and (ii) at least one anion.

In some embodiments, the tetrathiafulvalene-2,3,6,7 tetrathiolate bridged bimetallic complex is a salt of (i) an oxidation product of a compound of Formula (I), wherein M is selected from the group consisting of Pt and Pd; R₁ and R₂ are independently selected from the group consisting of: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring; and wherein the oxidation product comprises a dication diradical, dication or cation of the compound, and (ii) at least one anion selected from the group consisting of borates, phosphates, triflates and antimonates; and wherein the at least one anion is optionally covalently bound to at least one capping ligand.

In some embodiments, the tetrathiafulvalene-2,3,6,7 tetrathiolate bridged bimetallic complex is [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂, [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, or [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

In a third aspect, the present disclosure encompasses a kit including a dication diradical tetrathiafulvalene-2,3,6,7 tetrathiolate bridged bimetallic complex near infrared luminescence agent and a pharmaceutically acceptable carrier.

In a fourth aspect, the present disclosure encompasses a molecular qubit which is a tetrathiafulvalene-2,3,6,7 tetrathiolate bridged bimetallic complex.

In some embodiments, the tetrathiafulvalene-2,3,6,7 tetrathiolate bridged bimetallic complex is a compound of Formula (I):

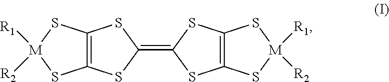

wherein M is a metal; R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring, and salts thereof.

In some embodiments, the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

In some embodiments, the tetrathiafulvalene-2,3,6,7 tetrathiolate bridged bimetallic complex is a salt having (i) an oxidation product of a compound of Formula (I), wherein the oxidation product comprises a dication diradical, dication or cation of the compound, and (ii) at least one anion.

In some embodiments, the tetrathiafulvalene-2,3,6,7 tetrathiolate bridged bimetallic complex is a salt of (i) an oxidation product of a compound of Formula (I), wherein M is selected from the group consisting of Pt and Pd; R₁ and R₂ are independently selected from the group consisting of: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring; and wherein the oxidation product comprises a dication diradical, dication or cation of the compound, and (ii) at least one anion selected from the group consisting of borates, phosphates, triflates and antimonates; and wherein the at least one anion is optionally covalently bound to at least one capping ligand.

In some embodiments, the tetrathiafulvalene-2,3,6,7 tetrathiolate bridged bimetallic complex is [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂, [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, or [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

In a fifth aspect, the present disclosure encompasses a method for cellular imaging. In some embodiments, the method includes administering a tetrathiafulvalene-2,3,6,7 tetrathiolate bridged bimetallic qubit to a cell comprising at least one cellular protein; permitting coordination of the tetrathiafulvalene-2,3,6,7 tetrathiolate bridged bimetallic qubit with the cellular protein to form a tetrathiafulvalene-2,3,6,7 tetrathiolate bridged bimetallic qubit-cellular protein coordination complex; illuminating the cell with a light source emitting light of at least one wavelength or wavelength band causing luminescence of the tetrathiafulvalene-2,3,6,7 tetrathiolate bridged bimetallic qubit-cellular protein coordination complex; and detecting luminescence with an image detector to visualize the cellular protein.

In some embodiments, the tetrathiafulvalene-2,3,6,7 tetrathiolate bridged bimetallic complex is a compound of Formula (I):

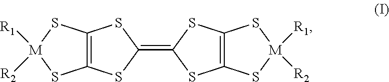

wherein M is a metal; R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring, and salts thereof.

In some embodiments, the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

In some embodiments, the tetrathiafulvalene-2,3,6,7 tetrathiolate bridged bimetallic complex is a salt having (i) an oxidation product of a compound of Formula (I), wherein the oxidation product comprises a dication diradical, dication or cation of the compound, and (ii) at least one anion.

In some embodiments, the tetrathiafulvalene-2,3,6,7 tetrathiolate bridged bimetallic complex is a salt of (i) an oxidation product of a compound of Formula (I), wherein M is selected from the group consisting of Pt and Pd; R₁ and R₂ are independently selected from the group consisting of: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring; and wherein the oxidation product comprises a dication diradical, dication or cation of the compound, and (ii) at least one anion selected from the group consisting of borates, phosphates, triflates and antimonates; and wherein the at least one anion is optionally covalently bound to at least one capping ligand.

In some embodiments, the tetrathiafulvalene-2,3,6,7 tetrathiolate bridged bimetallic complex is [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂. [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, or [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

In a sixth aspect, the present disclosure encompasses a chromophoric composition including an oxidation product of a coordination complex of Formula (I):

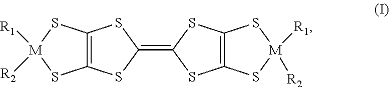

wherein M is a metal; R₁ and R₂ are each independently a therapeutic moiety, optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring; wherein said oxidation product is a dication diradical of the coordination complex, and (ii) at least one anion.

In some embodiments, the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

In some embodiments, the therapeutic moiety is -L-T, wherein L is a linking moiety and T is a biologically active agent.

In some embodiments, at least one of R₁ and R₂ is a therapeutic moiety.

In some embodiments, R₁ and R₂ are taken together to form a substituted ring, and the therapeutic moiety is the substituent of the ring.

In some embodiments, the linking moiety is independently selected from: —O—, —SO₂—, —NH—, —C(O)—, —C(O)O—, —OC(O)—, optionally substituted alkyl, optionally substituted alkylene, optionally substituted haloalkylene, optionally substituted hydroxyalkylene, optionally substituted alkyleneoxy, optionally substituted heteroalkylene, optionally substituted arylene, optionally substituted aryleneoxy, optionally substituted heterocyclyl and optionally substituted heterocyclyldiyl.

In some embodiments, the biologically active agent is an antineoplastic, antibiotic, corticosteroid, cytotoxic, or immunosuppressive drug.

In some embodiments, the biologically active agent is a protein, antibody, antibody fragment, peptide, aptamer, oligomer, ribonucleic acid or deoxyribonucleic acid.

In a seventh aspect, the present disclosure encompasses a pharmaceutical composition including a chromophoric composition of an oxidation product of a coordination complex of Formula (I):

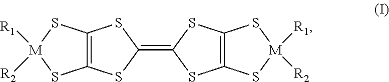

wherein M is a metal; R₁ and R₂ are each independently a therapeutic moiety, optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring; wherein said oxidation product is a dication diradical of the coordination complex, and (ii) at least one anion; and a pharmaceutically acceptable carrier.

In an eighth aspect, the present disclosure encompasses a compound of Formula (I):

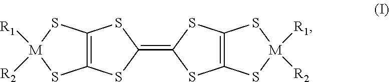

wherein M is selected from Pt and Pd; R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring, and salts thereof.

In some embodiments, M is Pt.

In some embodiments, M is Pd.

In some embodiments, R₁ and R₂ are taken together to form an optionally substituted ring.

In some embodiments, R₁ and R₂ taken together to form a ring is 1,2-bis(diphenylphosphino)ethyl or 1,5-cyclooctadiene.

In some embodiments, R₁ and R₂ are each tris(4-trifluoromethylphenyl)phosphine.

In a ninth aspect, the present disclosure encompasses a composition of (i) an oxidation product of a coordination complex of Formula (I):

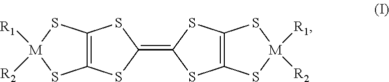

wherein M is selected from Pt and Pd; R₁ and R₂ are independently selected from: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring, wherein said oxidation product is a dication diradical, dication or cation of the coordination complex, and (ii) at least one anion selected from borates, phosphates, triflates and antimonates; and wherein the at least one anion is optionally covalently bound to at least one capping ligand.

In some embodiments, M is Pt.

In some embodiments, M is Pd.

In some embodiments, R₁ and R₂ are taken together to form an optionally substituted ring.

In some embodiments, R₁ and R₂ taken together to form a ring is 1,2-bis(diphenylphosphino)ethyl or 1,5-cyclooctadiene.

In some embodiments, R₁ and R₂ are each tris(4-trifluoromethylphenyl)phosphine.

In a tenth aspect, the present disclosure encompasses the following compounds: is [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂, [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, or [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

In an eleventh aspect, the present disclosure encompasses a method of magnetic resonance imaging measurement of a target, which entails exposing the target to a diagnostically effective amount of a contrast agent comprising a tetrathiafulvalene-2,3,6,7 tetrathiolate bridged bimetallic complex to form an activated target; exposing the activated target to near infrared light of a frequency selected to excite nuclear or electron spin transitions of the contrast agent; and detecting magnetic resonance signals from the activated target or the surrounding environment.

In some embodiments, the method involves generating an image, dynamic flow data, diffusion data or perfusion data from detected magnetic resonance signals.

In some embodiments, the target is in vivo or in vitro.

In some embodiments, the target is an organ, a tissue or a cell.

In some embodiments, the cell is a stem cell, immune cell, blood cell, neuron or beta cell.

In some embodiments, the target is in an aqueous solution.

In some embodiments, the method also includes transferring polarization to the aqueous solution.

In some embodiments, the tetrathiafulvalene-2,3,6,7 tetrathiolate bridged bimetallic complex contrast agent is a compound of Formula (I):

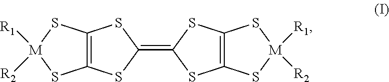

wherein M is a metal; and R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring, and salts thereof.

In some embodiments, the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

In some embodiments, the tetrathiafulvalene-2,3,6,7 tetrathiolate bridged bimetallic complex is (i) an oxidation product of the compound of Formula (I), wherein the oxidation product comprises a dication diradical, dication or cation of the compound, and (ii) at least one anion.

In some embodiments, the tetrathiafulvalene-2,3,6,7 tetrathiolate bridged bimetallic complex is (i) an oxidation product of the compound of Formula (I), wherein M is selected from the group consisting of Pt and Pd; and R₁ and R₂ are independently selected from the group consisting of: optionally substituted alkylphosphino, optionally substituted alkyl, optionally substituted aralkyl, and optionally substituted aryl, and wherein R₁ and R₂ taken together may form an optionally substituted ring; and wherein the oxidation product comprises a dication diradical, dication or cation of the compound, and (ii) at least one anion selected from the group consisting of borates, phosphates, triflates and antimonates; and wherein the at least one anion is optionally covalently bound to at least one capping ligand.

In some embodiments, the tetrathiafulvalene-2,3,6,7 tetrathiolate bridged bimetallic complex is [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂, [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

In some embodiments, the method also includes dynamic nuclear polarization.

These and other aspects are described further below with reference to the drawings.

### BRIEF DESCRIPTION OF THE DRAWINGS

FIG. 1 is a schematic diagram showing various electronic states of the tetrathiafulavalene-2,3,6,7-tetrathiolate (TTFtt) bridging structure.

FIG. 2 is a schematic diagram showing exemplary routes to the synthesis of tin and nickel complexes with tetrathiafulavalene-2,3,6,7-tetrathiolate as a bridging ligand in accordance with certain disclosed embodiments.

FIG. 3 is a schematic diagram showing transmetallation of a tin tetrathiafulavalene-2,3,6,7-tetrathiolate to form a Pd or Pt TTFtt dication diradical in accordance with certain disclosed embodiments.

FIG. 4 is a depiction of tetrathiafulavalene-2,3,6,7-tetrathiolate dication diradical compounds and graphs of the UV-vis-NIR spectra associated with each compound in accordance with certain disclosed embodiments.

FIG. 5 is a plot of the absorption and emission spectra for [(Pd{dppe})₂TTFtt][BAr^{F}₄]₂ (PdTTFtt; compound 1) in dichloromethane at 298K in accordance with certain disclosed embodiments.

FIG. 6 is a plot of the absorption and emission spectra for [(Pt{dppe})₂TTFtt][BAr^{F}₄]₂ (PtTTFtt; compound 2) in dichoromethane at 298K in accordance with certain disclosed embodiments.

FIG. 7 is a plot of the absorption and emission spectra for [(Pt{P(4-CF₃Ph)₃}₂): TTFtt][BAr^{F}₄]₂ (PtCF₃TTFtt; compound 3) in dichloromethane at 298K in accordance with certain disclosed embodiments.

FIG. 8A is plot of UV-Vis-NIR and photoluminescence of compound 5 in acetonitrile at 298 K in accordance with certain disclosed embodiments.

FIG. 8B is a plot of UV-Vis-NIR and photoluminescence of compound 6 in dichloromethane (DCM) at 298 K in accordance with certain disclosed embodiments.

FIG. 9 is a plot of UV-Vis-NIR and photoluminescence of compound 7 in DCM at 298 K in accordance with certain disclosed embodiments.

FIG. 10 is a plot of the photoluminescence quantum yield (PLQY) for [(Pt{dppe})₂TTFtt][BAr^{F}₄]₂ (PtTTFtt; compound 2) in comparison to a conventional near IR dye 4-(7-(2-phenyl-4H-1-benzothiopyran-4-ylidene)-4-chloro-3,5-trimethylene-1,3,5-heptatrienyl)-2-phenyl-1-benzothiopyrylium perchlorate (IR 26, available from Exciton of Dayton, Ohio). IR 26 was determined to have PLQY=0.05% in oxygenated 1,2-dichloroethane, while PtTTFtt had PLQY of 0.09% in accordance with certain disclosed embodiments.

FIG. 11 is a plot of the photoluminescence quantum yield (PLQY) for compounds 1-3 at various concentrations as measured in dichloromethane at 298K under anaerobic conditions in accordance with certain disclosed embodiments.

FIG. 12 is a plot of the cyclic voltammogram of 1 mM PtTTFtt (compound 2) monocation with 0.1 M [tetrabutylammonium][PF₆] electrolyte in dichloromethane in accordance with certain disclosed embodiments.

FIG. 13 is a plotted comparison of relative Rabi frequencies of compound 2 and corresponding monocation monoradical complex in DCM at room temperature in accordance with certain disclosed embodiments.

FIG. 14 is a set of graphs depicting the redox-switchable photoluminescence of PtTTFtt (compound 2) in dichloromethane in accordance with certain disclosed embodiments.

FIG. 15A is a graph depicting spin manipulation with pulsed electron paramagnetic resonance for PtTTFtt (compound 2) in accordance with certain disclosed embodiments.

FIG. 15B is a graph of nutation frequency plotted against the magnetic field for PtTTFtt (compound 2) in accordance with certain disclosed embodiments.

FIG. 16A is plot illustrating a spin inversion recovery measurement of compound 2 in DCM at room temperature, (data points represented by circles), fit to a monoexponential decay, (represented by solid line), in accordance with certain disclosed embodiments.

FIG. 16B is a plot illustrating the Hahn echo decay measurement of compound 2 in DCM at room temperature, (data points represented by circles), fit to a monoexponential decay, (represented by solid line), in accordance with certain disclosed embodiments.

FIG. 17A is a depiction of (Pt{1,5-cyclooctadiene})₂ tetrathiafulvalene-2,3,6,7 tetrathiolate dication diradical (compound 4, PtCODTTFtt) in accordance with certain disclosed embodiments.

FIG. 17B is an ¹H NMR spectrum indicating diradical character of compound 4 in accordance with certain disclosed embodiments.

FIG. 17C is an ¹H NMR spectrum (Evans method) demonstrating paramagnetism of compound 4 in accordance with certain disclosed embodiments.

FIG. 18 is a depiction of a graph of the UV-vis-NIR spectrum associated with compound 4 in dichloromethane (DCM) in accordance with certain disclosed embodiments.

FIG. 19 is a depiction of a graph of the UV-vis-NIR spectrum associated with compound 4 in MeCN (acetonitrile) in accordance with certain disclosed embodiments.

FIG. 20 is a plot of the cyclic voltammogram of 1 mM PtCODTTFtt (compound 4) in dichloromethane in accordance with certain disclosed embodiments.

FIG. 21 is a cell viability assay of compound 4 after 72 hr treatment, using 1% DMSO solution in accordance with certain disclosed embodiments.

FIG. 22 is a plot demonstrating the reactivity of compound 4 in the presence of small nucleophiles in accordance with certain disclosed embodiments.

FIG. 23 is a plot demonstrating the reactivity of compound 4 in the presence of ubiquitin in accordance with certain disclosed embodiments.

FIG. 24A is the single crystal structure of compound 4 in 1:2 acetonitrile:water in accordance with certain disclosed embodiments.

FIG. 24B is the single crystal structure of compound 5 in DCM in accordance with certain disclosed embodiments.

FIG. 24C is a single crystal structure of compound 7 in DCM in accordance with certain disclosed embodiments.

FIG. 25A is a single crystal structure of compound 8 in DCM in accordance with certain disclosed embodiments.

FIG. 25B is a single crystal structure of compound 9 in DCM in accordance with certain disclosed embodiments.

### DETAILED DESCRIPTION

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

The present tetrathiafulvalene-2,3,6,7-tetrathiolate (TTFtt) molecules exhibit remarkable photophysical and magnetic properties due to their unique electronic structure. They are synthetically tunable, air and moisture stable and exhibit long room temperature lifetimes.

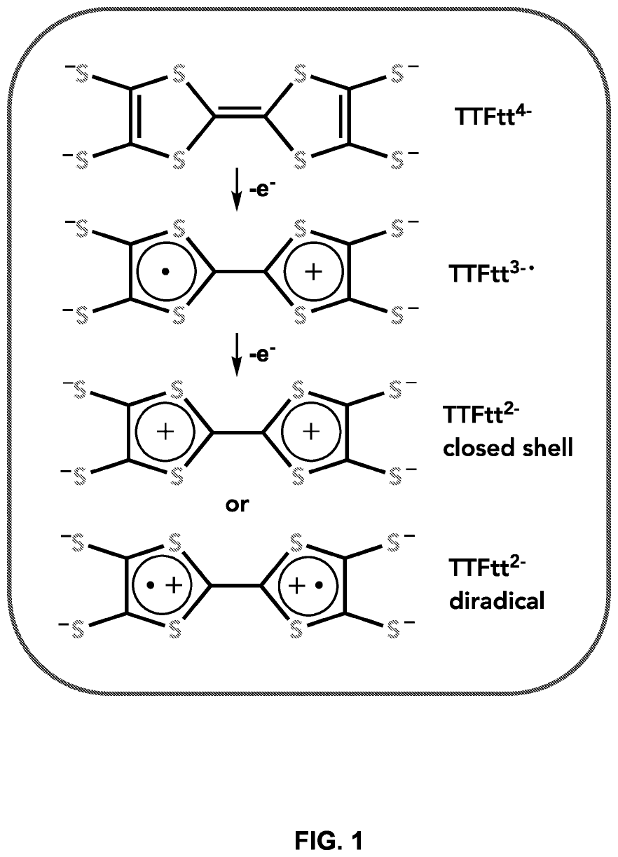

As illustrated in FIG. 1, the tetrathiafulvalene-2,3,6,7-tetrathiolate bridging structure may be a radical cation species, a closed shell dication or a diradical dication depending upon the conditions and/or features of the particular metals and ligands selected for the ends of the molecule. The diradical may be a diradical singlet or a diradical triplet.

### Definitions

As used herein, the term “about” means+/−10% of any recited value. As used herein, this term modifies any recited value, range of values, or endpoints of one or more ranges.

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Such an aliphatic can be unsubstituted or substituted with one or more groups, such as groups described herein for an alkyl group.

The term “acyl,” or “alkanoyl,” as used interchangeably herein, represents an alkyl group, as defined herein, or hydrogen attached to the parent molecular group through a carbonyl group, as defined herein. This group is exemplified by formyl, acetyl, propionyl, butanoyl, and the like. The alkanoyl group can be substituted or unsubstituted. For example, the alkanoyl group can be substituted with one or more substitution groups, as described herein for alkyl. In some embodiments, the unsubstituted acyl group is a C₂₋₇ acyl or alkanoyl group. In particular embodiments, the alkanoyl group is —C(O)-Ak, in which Ak is an alkyl group, as defined herein.

By “alkoxy” is meant-OR, where R is an optionally substituted alkyl group, as described herein. Exemplary alkoxy groups include methoxy, ethoxy, butoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkoxyalkyl” is meant an alkyl group, as defined herein, which is substituted with an alkoxy group, as defined herein. Exemplary unsubstituted alkoxyalkyl groups include between 2 to 12 carbons (C₂₋₁₂ alkoxyalkyl), as well as those having an alkyl group with 1 to 6 carbons and an alkoxy group with 1 to 6 carbons (i.e., C₁₋₆ alkoxy-C₁₋₆ alkyl).

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₁₋₆ alkoxy (e.g., —O-Ak, wherein Ak is optionally substituted C₁₋₆ alkyl); (2) C₁₋₆ alkylsulfinyl (e.g., —S(O)-Ak, wherein Ak is optionally substituted C₁₋₆ alkyl); (3) C₁₋₆ alkylsulfonyl (e.g., —SO₂-Ak, wherein Ak is optionally substituted C₁₋₆ alkyl); (4) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —O-L-Ar, wherein L is a bivalent form of optionally substituted alkyl and Ar is optionally substituted aryl); (7) aryloyl (e.g., —C(O)—Ar, wherein Ar is optionally substituted aryl); (8) azido (e.g., —N₃); (9) cyano (e.g., —CN); (10) carboxyaldehyde (e.g., —C(O) H); (11) C₃₋₈ cycloalkyl (e.g., a monovalent saturated or unsaturated non-aromatic cyclic C₃₋₈ hydrocarbon group); (12) halo (e.g., F, Cl, Br, or I); (13) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (14) heterocyclyloxy (e.g., —O-Het, wherein Het is heterocyclyl, as described herein); (15) heterocyclyloyl (e.g., —C(O)-Het, wherein Het is heterocyclyl, as described herein); (16) hydroxyl (e.g., —OH); (17) N-protected amino; (18) nitro (e.g., —NO₂); (19) oxo (e.g., ═O) or hydroxyimino (e.g., ═N—OH); (20) C₃₋₈ spirocyclyl (e.g., an alkylene or heteroalkylene diradical, both ends of which are bonded to the same carbon atom of the parent group); (21) C₁₋₆ thioalkoxy (e.g., —S-Ak, wherein Ak is optionally substituted C₁₋₆ alkyl); (22) thiol (e.g., —SH); (23) —CO₂R^{A}, where R^{A} is selected from the group consisting of (a) hydrogen, (b) C₁₋₆ alkyl, (c) C₄₋₁₈ aryl, and (d)(C₄₋₁₈ aryl) C₁₋₆ alkyl (e.g., -L-Ar, wherein L is a bivalent form of optionally substituted alkyl group and Ar is optionally substituted aryl); (24) —C(O)NR^{B}R^{C}, where each of R^{B} and R^{C} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); (25) —SO₂R^{D}, where R^{D} is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl group and Ar is optionally substituted aryl); (26) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl group and Ar is optionally substituted aryl); and (27) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., L-Ar, wherein L is a bivalent form of optionally substituted alkyl group and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl group and Cy is optionally substituted cycloalkyl, as described herein), wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a multivalent (e.g., bivalent, trivalent, tetravalent, etc.) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can be saturated or unsaturated (e.g., having one or more double bonds or triple bonds). The alkylene group can also be substituted or unsubstituted. For example, the alkylene group can be substituted with one or more substitution groups, as described herein for alkyl. In one instance, a substituted alkylene group can include an optionally substituted haloalkylene (e.g., an optionally substituted alkylene substituted with one or more hydroxyl groups, as defined herein), an optionally substituted haloalkylene (e.g., an optionally substituted alkylene substituted with one or more halo groups, as defined herein), and the like.

By “alkyleneoxy” is meant an alkylene group, as defined herein, attached to the parent molecular group through an oxygen atom.

By “amino” is meant-NR^{N1}R^{N2}, where each of R^{N1} and R^{N2} is, independently, H, optionally substituted alkyl, optionally substituted cycloalkyl, or optionally substituted aryl; or R^{N1} and R^{N2}, taken together with the nitrogen atom to which each are attached, form an optionally substituted heterocyclyl group or heterocycle, as defined herein; or R^{N1} and R^{N2}, taken together, form an optionally substituted alkylene or heteroalkylene (e.g., as described herein).

By “aminoalkyl” is meant an alkyl group, as defined herein, substituted by an amino group, as defined herein. Non-limiting aminoalkyl groups include-L-NR^{N1}R^{N2}, where L is a multivalent alkyl group, as defined herein; each of R^{N1} and R^{N2} is, independently, H, optionally substituted alkyl, or optionally substituted aryl; or R^{N1} and R^{N2}, taken together with the nitrogen atom to which each are attached, form a heterocyclyl group, as defined herein.

By “ammonium” is meant a group including a protonated nitrogen atom N⁺. Exemplary ammonium groups include-N+R^{N1}R^{N2}R^{N3} where each of R^{N1}, R^{N2}, and R^{N3} is, independently, H, optionally substituted alkyl, optionally substituted cycloalkyl, or optionally substituted aryl; or R^{N1} and R^{N2}, taken together with the nitrogen atom to which each are attached, form an optionally substituted heterocyclyl group or heterocycle; or R^{N1} and R^{N2}, taken together, form an optionally substituted alkylene or heteroalkylene (e.g., as described herein); or R^{N1} and R^{N2} and R^{N3}, taken together with the nitrogen atom to which each are attached, form an optionally substituted heterocyclyl group or heterocycle, such as a heterocyclic cation.

By “anion” is meant any suitable counterion. In some embodiments, the anionic species is a borate anion comprising four aryl or substituted aryl groups (e.g. a fluoroaryl or a (perfluoroalkyl) aryl group), such as, but not limited to tetrakis(3,5-bis(trifluoromethyl)phenyl borate. In some embodiments, the anion is hexafluorophosphate. Other anionic species include, but are not limited to, triflate or hexafluoroantimonate.

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Huckel rule (4n+2). The point of attachment to the parent structure typically is through an aromatic portion of the condensed ring system. Such an aromatic can be unsubstituted or substituted with one or more groups, such as groups described herein for an alkyl or aryl group. Yet other substitution groups can include aliphatic, haloaliphatic, halo, nitrate, cyano, sulfonate, sulfonyl, or others.

By “aryl” is meant a group that contains any carbon-based aromatic group including, but not limited to, phenyl, benzyl, anthracenyl, anthryl, benzocyclobutenyl, benzocyclooctenyl, biphenylyl, chrysenyl, dihydroindenyl, fluoranthenyl, indacenyl, indenyl, naphthyl, phenanthryl, phenoxybenzyl, picenyl, pyrenyl, terphenyl, and the like, including fused benzo-C₄₋₈ cycloalkyl radicals (e.g., as defined herein) such as, for instance, indanyl, tetrahydronaphthyl, fluorenyl, and the like. The term aryl also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O)-Ak, wherein Ak is optionally substituted C₁-6 alkyl); (2) C₁₋₆ alkyl; (3) C₁₋₆ alkoxy (e.g., —O-Ak, wherein Ak is optionally substituted C₁₋₆ alkyl); (4) C₁₋₆ alkoxy-C₁₋₆ alkyl (e.g., -L-O-Ak, wherein L is a bivalent form of optionally substituted alkyl group and Ak is optionally substituted C₁₋₆ alkyl); (5) C₁₋₆ alkylsulfinyl (e.g., —S(O)-Ak, wherein Ak is optionally substituted C₁₋₆ alkyl); (6) C₁₋₆ alkylsulfinyl-C₁₋₆ alkyl (e.g., -L-S(O)-Ak, wherein L is a bivalent form of optionally substituted alkyl group and Ak is optionally substituted C₁₋₆ alkyl); (7) C₁₋₆ alkylsulfonyl (e.g., —SO₂-Ak, wherein Ak is optionally substituted C₁₋₆ alkyl); (8) C₁₋₆ alkylsulfonyl-C₁₋₆ alkyl (e.g., -L-SO₂-Ak, wherein L is a bivalent form of optionally substituted alkyl group and Ak is optionally substituted C₁₋₆ alkyl); (9) aryl; (10) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (11) C₁₋₆ aminoalkyl (e.g., an alkyl group, as defined herein, substituted by one or more-NR^{N1}R^{N2} groups, as described herein); (12) heteroaryl (e.g., a subset of heterocyclyl groups (e.g., a 5-, 6- or 7-membered ring, unless otherwise specified, containing one, two, three, or four non-carbon heteroatoms), which are aromatic); (13) (C₄₋₁₈ aryl) C₁₋₆ alkyl (e.g., -L-Ar, wherein L is a bivalent form of optionally substituted alkyl and Ar is optionally substituted aryl); (14) aryloyl (e.g., —C(O)—Ar, wherein Ar is optionally substituted aryl); (15) azido (e.g., —N₃); (16) cyano (e.g., —CN); (17) C₁₋₆ azidoalkyl (e.g., an alkyl group, as defined herein, substituted by one or more azido groups, as described herein); (18) carboxyaldehyde (e.g., —C(O) H); (19) carboxyaldehyde-C₁₋₆ alkyl (e.g., an alkyl group, as defined herein, substituted by one or more carboxyaldehyde groups, as described herein); (20) C₃₋₈ cycloalkyl (e.g., a monovalent saturated or unsaturated non-aromatic cyclic C₃₋₈ hydrocarbon group); (21) (C₃₋₈ cycloalkyl) C₁₋₆ alkyl (e.g., an alkyl group, as defined herein, substituted by one or more cycloalkyl groups, as described herein); (22) halo (e.g., F, Cl, Br, or I); (23) C₁₋₆ haloalkyl (e.g., an alkyl group, as defined herein, substituted by one or more halo groups, as described herein); (24) heterocyclyl (e.g., a 5-, 6- or 7-membered ring, unless otherwise specified, containing one, two, three, or four non-carbon heteroatoms, such as nitrogen, oxygen, phosphorous, sulfur, or halo); (25) heterocyclyloxy (e.g., —O-Het, wherein Het is heterocyclyl, as described herein); (26) heterocyclyloyl (e.g., —C(O)-Het, wherein Het is heterocyclyl, as described herein); (27) hydroxyl (e.g., —OH); (28) C₁₋₆ hydroxyalkyl (e.g., an alkyl group, as defined herein, substituted by one or more hydroxyl, as described herein); (29) nitro (e.g., —NO₂); (30) C₁₋₆ nitroalkyl (e.g., an alkyl group, as defined herein, substituted by one or more nitro, as described herein); (31) N-protected amino; (32) N-protected amino-C₁₋₆ alkyl (e.g., an alkyl group, as defined herein, substituted by one or more N-protected amino groups); (33) oxo (e.g., ═O) or hydroxyimino (e.g., ═N—OH); (34) C₁₋₆ thioalkoxy (e.g., —S-Ak, wherein Ak is optionally substituted C₁₋₆ alkyl); (35) thio-C₁₋₆ alkoxy-C₁₋₆ alkyl (e.g., -L-S-Ak, wherein L is a bivalent form of optionally substituted alkyl and Ak is optionally substituted C₁₋₆ alkyl); (36) —(CH₂)ᵣCO₂R^{A}, where r is an integer of from zero to four, and R^{A} is selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (37) —(CH₂)ᵣCONR^{B}R^{C}, where r is an integer of from zero to four and where each R^{B} and R^{C} is independently selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (38) —(CH₂)SO₂R^{D}, where r is an integer of from zero to four and where R^{D} is selected from the group consisting of (a) C₁₋₆ alkyl, (b) C₄₋₁₈ aryl, and (c) (C₄₋₁₈ aryl) C₁₋₆ alkyl (e.g., -L-Ar, wherein L is a bivalent form of optionally substituted alkyl and Ar is optionally substituted aryl); (39) —(CH₂)ᵣSO₂NR^{E}R^{F}, where r is an integer of from zero to four and where each of R^{E} and R^{F} is, independently, selected from the group consisting of (a) hydrogen, (b) C₁₋₆ alkyl, (c) C₄₋₁₈ aryl, and (d) (C₄₋₁₈ aryl) C₁₋₆ alkyl (e.g., -L-Ar, wherein L is a bivalent form of optionally substituted alkyl and Ar is optionally substituted aryl); (40) —(CH₂)ᵣNR^{G}R^{H}, where r is an integer of from zero to four and where each of R^{G} and R^{H} is, independently, selected from the group consisting of (a) hydrogen, (b) an N-protecting group, (c) C₁₋₆ alkyl, (d) C₂₋₆ alkenyl (e.g., optionally substituted alkyl having one or more double bonds), (e) C₂₋₆ alkynyl (e.g., optionally substituted alkyl having one or more triple bonds), (f) C₄₋₁₈ aryl, (g) (C₄₋₁₈ aryl) C₁₋₆ alkyl (e.g., -L-Ar, wherein L is a bivalent form of optionally substituted alkyl and Ar is optionally substituted aryl), (h) C₃₋₈ cycloalkyl, and (i) (C₃₋₈ cycloalkyl) C₁₋₆ alkyl (e.g., -L-Cy, wherein L is a bivalent form of optionally substituted alkyl and Cy is optionally substituted cycloalkyl, as described herein), wherein in one embodiment no two groups are bound to the nitrogen atom through a carbonyl group or a sulfonyl group; (41) thiol (e.g., —SH); (42) perfluoroalkyl (e.g., an alkyl group having each hydrogen atom substituted with a fluorine atom); (43) perfluoroalkoxy (e.g., —OR^{f}, where R^{f} is an alkyl group having each hydrogen atom substituted with a fluorine atom); (44) aryloxy (e.g., —OAr, where Ar is optionally substituted aryl); (45) cycloalkoxy (e.g., —O-Cy, wherein Cy is optionally substituted cycloalkyl, as described herein); (46) cycloalkylalkoxy (e.g., —O-L-Cy, wherein L is a bivalent form of optionally substituted alkyl and Cy is optionally substituted cycloalkyl, as described herein); and (47) arylalkoxy (e.g., —O-L-Ar, wherein L is a bivalent form of optionally substituted alkyl and Ar is optionally substituted aryl). In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “arylalkoxy” is meant an arylalkylene group, as defined herein, attached to the parent molecular group through an oxygen atom. In some embodiments, the arylalkoxy group is —O-Ak-Ar, in which Ak is an optionally substituted alkylene, as defined herein, and Ar is an optionally substituted aryl, as defined herein.

By “(aryl)(alkyl) ene” is meant a bivalent form including an arylene group, as described herein, attached to an alkylene or a heteroalkylene group, as described herein. In some embodiments, the (aryl)(alkyl) ene group is -L-Ar- or -L-Ar-L- or -Ar-L-, in which Ar is an arylene group and each L is, independently, an optionally substituted alkylene group or an optionally substituted heteroalkylene group.

By “arylalkylene” is meant an aryl group, as defined herein, attached to the parent molecular group through an alkylene group, as defined herein. In some embodiments, the arylalkylene group is -Ak-Ar, in which Ak is an optionally substituted alkylene, as defined herein, and Ar is an optionally substituted aryl, as defined herein. The arylalkylene group can be substituted or unsubstituted. For example, the arylalkylene group can be substituted with one or more substitution groups, as described herein for aryl and/or alkyl. Exemplary unsubstituted arylalkylene groups are of from 7 to 16 carbons (C₇₋₁₆ arylalkylene), as well as those having an aryl group with 4 to 18 carbons and an alkylene group with 1 to 6 carbons (i.e., (C₄₋₁₈ aryl) C₁₋₆ alkylene).

By “arylene” is meant a multivalent (e.g., bivalent, trivalent, tetravalent, etc.) form of an aryl group, as described herein. Exemplary arylene groups include phenylene, naphthylene, biphenylene, triphenylene, diphenyl ether, acenaphthenylene, anthrylene, or phenanthrylene. In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for aryl.

By “aryleneoxy” is meant an arylene group, as defined herein, attached to the parent molecular group through an oxygen atom.

By “aryloxy” is meant an aryl group, as defined herein, attached to the parent molecular group through an oxygen atom.

By “aryloyl” is meant an aryl group that is attached to the parent molecular group through a carbonyl group. In some embodiments, an unsubstituted aryloyl group is a C₇₋₁₁ aryloyl or C₅₋₁₉ aryloyl group. In particular embodiments, the aryloyl group is —C(O)—Ar, in which Ar is an aryl group, as defined herein.

By “boranyl” is meant a —BR₂ group, in which each R, independently, can be H, halo, or optionally substituted alkyl.

By “borono” is meant a —BOH₂ group.

By “capping ligand” is meant a compound such as dipyridinylphosphinic acid sodium salt, 3,3′,3″-phosphanetris(benzenesulfonic acid) trisodium salt or 2,2′-bipyridine-4,4′-dicarboxylic acid disodium salt.

By “carboxyl” is meant a —CO₂H group.

By “carboxylate anion” is meant a —CO₂⁻ group.

By “chromophore” is meant a compound that absorbs light at a specific frequency and so imparts color to the compound.

By “coordination complex” or “complex” is meant a compound in which there is a coordinate bond between a metal ion and an electron pair donor, which can also be referred to herein as a “ligand” or “chelating group”. Thus, ligands or chelating groups are generally molecules or molecular ions having unshared electron pairs available for donation to a metal ion.

By “coordination bond” is meant an interaction between an electron pair donor and a coordination site on a metal ion resulting in an attractive force between the electron pair donor and the metal ion. The use of this term is not meant to be limiting, as certain coordinate bonds can also be classified as having more or less covalent character (if not entirely covalent character) depending on the characteristics of the metal ion and the electron pair donor.

By “counterion” is meant one or more charge balancing ions which may be cations, anions or zwitterions.

By “covalent bond” is meant a covalent bonding interaction between two components. Non-limiting covalent bonds include a single bond, a double bond, a triple bond, or a spirocyclic bond, in which at least two molecular groups are bonded to the same carbon atom.

By “coordination site” is meant a ligand's unshared electron pair, negative charge, atoms or functional groups capable of forming an unshared electron pair or negative charge.

By “cyano” is meant a —CN group.

By “cyclic group” or “cyclyl” as used herein to refer to either aryl groups, non-aryl groups (e.g., cycloalkyl or heterocycloalkyl groups), or both. Cyclic groups have one or more ring systems that can be substituted or unsubstituted. A cyclic group can contain one or more aryl groups, one or more non-aryl groups, or one or more aryl groups and one or more non-aryl groups.

By “cycloalkyl” is meant a monovalent saturated or unsaturated non-aromatic cyclic hydrocarbon group of from three to ten carbons (e.g., C₃₋₈ or C₃₋₁₀), unless otherwise specified, and is exemplified by cyclopropyl, cyclobutyl, cyclopentyl, cyclohexyl, cycloheptyl, bicyclo[2.2.1.]heptyl, and the like. The term cycloalkyl also includes “cycloalkenyl,” which is defined as a non-aromatic carbon-based ring composed of three to ten carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The cycloalkyl group can also be substituted or unsubstituted. For example, the cycloalkyl group can be substituted with one or more groups including those described herein for alkyl.

By “DCM” is meant dichloromethane.

By “luminescence” is meant the emission of light by a substance that has not been heated.

By “halo” is meant F, Cl, Br, or I.

By “haloalkyl” is meant an alkyl group, as defined herein, substituted with one or more halo.

By “haloalkylene” is meant an alkylene group, as defined herein, substituted with one or more halo.

By “heteroaliphatic” is meant an aliphatic group, as defined herein, including at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

By “heteroalkyl” is meant an alkyl group, as defined herein, containing one, two, three, or four non-carbon heteroatoms (e.g., independently selected from the group consisting of nitrogen, oxygen, phosphorous, sulfur, selenium, or halo).

By “heteroalkylene” is meant an alkylene group, as defined herein, containing one, two, three, or four non-carbon heteroatoms (e.g., independently selected from the group consisting of nitrogen, oxygen, phosphorous, sulfur, selenium, or halo). The heteroalkylene group can be saturated or unsaturated (e.g., having one or more double bonds or triple bonds). The heteroalkylene group can be substituted or unsubstituted. For example, the heteroalkylene group can be substituted with one or more substitution groups, as described herein for alkyl.

By “heteroaryl” is meant a subset of heterocyclyl groups, as defined herein, which are aromatic, i.e., they contain 4n+2 pi electrons within the mono- or multicyclic ring system.

The term “heterocycloalkyl” is a type of cycloalkyl group as defined above where at least one of the carbon atoms and its attached hydrogen atoms, if any, are replaced by O, S, N, or NH. The heterocycloalkyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, sulfonic acid, sulfinic acid, fluoroacid, phosphonic acid, ester, ether, halide, hydroxy, ketone, nitro, cyano, azido, silyl, sulfonyl, sulfinyl, or thiol, as described herein.

By “heterocycle” is meant a compound having one or more heterocyclyl moieties. Non-limiting heterocycles include optionally substituted imidazole, optionally substituted triazole, optionally substituted tetrazole, optionally substituted pyrazole, optionally substituted imidazoline, optionally substituted pyrazoline, optionally substituted imidazolidine, optionally substituted pyrazolidine, optionally substituted pyrrole, optionally substituted pyrroline, optionally substituted pyrrolidine, optionally substituted tetrahydrofuran, optionally substituted furan, optionally substituted thiophene, optionally substituted oxazole, optionally substituted isoxazole, optionally substituted isothiazole, optionally substituted thiazole, optionally substituted oxathiolane, optionally substituted oxadiazole, optionally substituted thiadiazole, optionally substituted sulfolane, optionally substituted succinimide, optionally substituted thiazolidinedione, optionally substituted oxazolidone, optionally substituted hydantoin, optionally substituted pyridine, optionally substituted piperidine, optionally substituted pyridazine, optionally substituted piperazine, optionally substituted pyrimidine, optionally substituted pyrazine, optionally substituted triazine, optionally substituted pyran, optionally substituted pyrylium, optionally substituted tetrahydropyran, optionally substituted dioxine, optionally substituted dioxane, optionally substituted dithiane, optionally substituted trithiane, optionally substituted thiopyran, optionally substituted thiane, optionally substituted oxazine, optionally substituted morpholine, optionally substituted thiazine, optionally substituted thiomorpholine, optionally substituted cytosine, optionally substituted thymine, optionally substituted uracil, optionally substituted thiomorpholine dioxide, optionally substituted indene, optionally substituted indoline, optionally substituted indole, optionally substituted isoindole, optionally substituted indolizine, optionally substituted indazole, optionally substituted benzimidazole, optionally substituted azaindole, optionally substituted azaindazole, optionally substituted pyrazolopyrimidine, optionally substituted purine, optionally substituted benzofuran, optionally substituted isobenzofuran, optionally substituted benzothiophene, optionally substituted benzisoxazole, optionally substituted anthranil, optionally substituted benzisothiazole, optionally substituted benzoxazole, optionally substituted benzthiazole, optionally substituted benzthiadiazole, optionally substituted adenine, optionally substituted guanine, optionally substituted tetrahydroquinoline, optionally substituted dihydroquinoline, optionally substituted dihydroisoquinoline, optionally substituted quinoline, optionally substituted isoquinoline, optionally substituted quinolizine, optionally substituted quinoxaline, optionally substituted phthalazine, optionally substituted quinazoline, optionally substituted cinnoline, optionally substituted naphthyridine, optionally substituted pyridopyrimidine, optionally substituted pyridopyrazine, optionally substituted pteridine, optionally substituted chromene, optionally substituted isochromene, optionally substituted chromenone, optionally substituted benzoxazine, optionally substituted quinolinone, optionally substituted isoquinolinone, optionally substituted carbazole, optionally substituted dibenzofuran, optionally substituted acridine, optionally substituted phenazine, optionally substituted phenoxazine, optionally substituted phenothiazine, optionally substituted phenoxathiine, optionally substituted quinuclidine, optionally substituted azaadamantane, optionally substituted dihydroazepine, optionally substituted azepine, optionally substituted diazepine, optionally substituted oxepane, optionally substituted thiepine, optionally substituted thiazepine, optionally substituted azocane, optionally substituted azocine, optionally substituted thiocane, optionally substituted azonane, optionally substituted azecine, etc. Optional substitutions include any described herein for aryl. Heterocycles can also include cations and/or salts of any of these (e.g., any described herein, such as optionally substituted piperidinium, optionally substituted pyrrolidinium, optionally substituted pyrazolium, optionally substituted imidazolium, optionally substituted pyridinium, optionally substituted quinolinium, optionally substituted isoquinolinium, optionally substituted acridinium, optionally substituted phenanthridinium, optionally substituted pyridazinium, optionally substituted pyrimidinium, optionally substituted pyrazinium, optionally substituted phenazinium, or optionally substituted morpholinium).

By “heterocyclyl” is meant a 3-, 4-, 5-, 6- or 7-membered ring (e.g., a 5-, 6- or 7-membered ring), unless otherwise specified, containing one, two, three, or four non-carbon heteroatoms (e.g., independently selected from the group consisting of nitrogen, oxygen, phosphorous, sulfur, selenium, or halo). The 3-membered ring has zero to one double bonds, the 4- and 5-membered ring has zero to two double bonds, and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include acridinyl, adenyl, alloxazinyl, azaadamantanyl, azabenzimidazolyl, azabicyclononyl, azacycloheptyl, azacyclooctyl, azacyclononyl, azahypoxanthinyl, azaindazolyl, azaindolyl, azecinyl, azepanyl, azepinyl, azetidinyl, azetyl, aziridinyl, azirinyl, azocanyl, azocinyl, azonanyl, benzimidazolyl, benzisothiazolyl, benzisoxazolyl, benzodiazepinyl, benzodiazocinyl, benzodihydrofuryl, benzodioxepinyl, benzodioxinyl, benzodioxanyl, benzodioxocinyl, benzodioxolyl, benzofuranyl, benzophenazinyl, benzodithiepinyl, benzodithiinyl, benzodioxocinyl, benzopyranonyl, benzopyranyl, benzopyrenyl, benzopyronyl, benzoquinolinyl, benzoquinolizinyl, benzothiadiazepinyl, benzothiadiazolyl, benzothiazepinyl, benzothiazocinyl, benzothiazolyl, benzothienyl, benzothiophenyl, benzothiazinonyl, benzothiazinyl, benzothiopyranyl, benzothiopyronyl, benzotriazepinyl, benzotriazinonyl, benzotriazinyl, benzotriazolyl, benzoxathiinyl, benzotrioxepinyl, benzoxadiazepinyl, benzoxathiazepinyl, benzoxathiepinyl, benzoxathiocinyl, benzoxazepinyl, benzoxazinyl, benzoxazocinyl, benzoxazolinonyl, benzoxazolinyl, benzoxazolyl, benzylsultamyl benzylsultimyl, bipyrazinyl, bipyridinyl, carbazolyl (e.g., 4H-carbazolyl), carbolinyl (e.g., β-carbolinyl), chromanonyl, chromanyl, chromenyl, cinnolinyl, coumarinyl, cytdinyl, cytosinyl, decahydroisoquinolinyl, decahydroquinolinyl, diazabicyclooctyl, diazetyl, diaziridinethionyl, diaziridinonyl, diaziridinyl, diazirinyl, dibenzisoquinolinyl, dibenzoacridinyl, dibenzocarbazolyl, dibenzofuranyl, dibenzophenazinyl, dibenzopyranonyl, dibenzopyronyl (xanthonyl), dibenzoquinoxalinyl, dibenzothiazepinyl, dibenzothiepinyl, dibenzothiophenyl, dibenzoxepinyl, dihydroazepinyl, dihydroazetyl, dihydrofuranyl, dihydrofuryl, dihydroisoquinolinyl, dihydropyranyl, dihydropyridinyl, dihydroypyridyl, dihydroquinolinyl, dihydrothienyl, dihydroindolyl, dioxanyl, dioxazinyl, dioxindolyl, dioxiranyl, dioxenyl, dioxinyl, dioxobenzofuranyl, dioxolyl, dioxotetrahydrofuranyl, dioxothiomorpholinyl, dithianyl, dithiazolyl, dithienyl, dithiinyl, furanyl, furazanyl, furoyl, furyl, guaninyl, homopiperazinyl, homopiperidinyl, hypoxanthinyl, hydantoinyl, imidazolidinyl, imidazolinyl, imidazolyl, indazolyl (e.g., 1H-indazolyl), indolenyl, indolinyl, indolizinyl, indolyl (e.g., 1H-indolyl or 3H-indolyl), isatinyl, isatyl, isobenzofuranyl, isochromanyl, isochromenyl, isoindazoyl, isoindolinyl, isoindolyl, isopyrazolonyl, isopyrazolyl, isoxazolidiniyl, isoxazolyl, isoquinolinyl, isoquinolinyl, isothiazolidinyl, isothiazolyl, morpholinyl, naphthindazolyl, naphthindolyl, naphthiridinyl, naphthopyranyl, naphthothiazolyl, naphthothioxolyl, naphthotriazolyl, naphthoxindolyl, naphthyridinyl, octahydroisoquinolinyl, oxabicycloheptyl, oxauracil, oxadiazolyl, oxazinyl, oxaziridinyl, oxazolidinyl, oxazolidonyl, oxazolinyl, oxazolonyl, oxazolyl, oxepanyl, oxetanonyl, oxetanyl, oxetyl, oxtenayl, oxindolyl, oxiranyl, oxobenzoisothiazolyl, oxochromenyl, oxoisoquinolinyl, oxoquinolinyl, oxothiolanyl, phenanthridinyl, phenanthrolinyl, phenazinyl, phenothiazinyl, phenothienyl (benzothiofuranyl), phenoxathiinyl, phenoxazinyl, phthalazinyl, phthalazonyl, phthalidyl, phthalimidinyl, piperazinyl, piperidinyl, piperidonyl (e.g., 4-piperidonyl), pteridinyl, purinyl, pyranyl, pyrazinyl, pyrazolidinyl, pyrazolinyl, pyrazolopyrimidinyl, pyrazolyl, pyridazinyl, pyridinyl, pyridopyrazinyl, pyridopyrimidinyl, pyridyl, pyrimidinyl, pyrimidyl, pyronyl, pyrrolidinyl, pyrrolidonyl (e.g., 2-pyrrolidonyl), pyrrolinyl, pyrrolizidinyl, pyrrolyl (e.g., 2H-pyrrolyl), pyrylium, quinazolinyl, quinolinyl, quinolizinyl (e.g., 4H-quinolizinyl), quinoxalinyl, quinuclidinyl, selenazinyl, selenazolyl, selenophenyl, succinimidyl, sulfolanyl, tetrahydrofuranyl, tetrahydrofuryl, tetrahydroisoquinolinyl, tetrahydroisoquinolyl, tetrahydropyridinyl, tetrahydropyridyl (piperidyl), tetrahydropyranyl, tetrahydropyronyl, tetrahydroquinolinyl, tetrahydroquinolyl, tetrahydrothienyl, tetrahydrothiophenyl, tetrazinyl, tetrazolyl, thiadiazinyl (e.g., 6H-1,2,5-thiadiazinyl or 2H,6H-1,5,2-dithiazinyl), thiadiazolyl, thianthrenyl, thianyl, thianaphthenyl, thiazepinyl, thiazinyl, thiazolidinedionyl, thiazolidinyl, thiazolyl, thienyl, thiepanyl, thiepinyl, thietanyl, thietyl, thiiranyl, thiocanyl, thiochromanonyl, thiochromanyl, thiochromenyl, thiodiazinyl, thiodiazolyl, thioindoxyl, thiomorpholinyl, thiophenyl, thiopyranyl, thiopyronyl, thiotriazolyl, thiourazolyl, thioxanyl, thioxolyl, thymidinyl, thyminyl, triazinyl, triazolyl, trithianyl, urazinyl, urazolyl, uretidinyl, uretinyl, uricyl, uridinyl, xanthenyl, xanthinyl, xanthionyl, and the like, as well as modified forms thereof (e.g., including one or more oxo and/or amino) and salts thereof. The heterocyclyl group can be substituted or unsubstituted. For example, the heterocyclyl group can be substituted with one or more substitution groups, as described herein for aryl.

By “heterocyclyldiyl” is meant a bivalent form of a heterocyclyl group, as described herein. In one instance, the heterocyclyldiyl is formed by removing a hydrogen from a heterocyclyl group. Exemplary heterocyclyldiyl groups include piperdylidene, quinolinediyl, etc. The heterocyclyldiyl group can also be substituted or unsubstituted. For example, the heterocyclyldiyl group can be substituted with one or more substitution groups, as described herein for heterocyclyl.

By “hydroxyl” is meant an —OH group.

By “hydroxyalkyl” is meant an alkyl group, as defined herein, substituted with one or more hydroxyl.

By “hydroxyalkylene” is meant an alkylene group, as defined herein, substituted with one or more hydroxy.

By “imaging” is meant a visualization technique which may be utilize imaging probes, spectroscopy or microscopy.

By “ligand” is meant a species, such as a molecule or ion, which interacts or binds in some way with another species. More particularly, as used herein, a “ligand” can refer to a molecule or ion that binds a metal ion in solution to form a coordination complex. The terms “ligand” and “chelating group” can be used interchangeably. The term “bridging ligand” can refer to a group that bonds to more than one metal ion or complex, thus providing a “bridge” between the metal ions or complexes. Organic bridging ligands can have two or more groups with unshared electron pairs separated by, for example, an alkylene or arylene group (i.e., a bivalent alkyl or aryl group). Groups with unshared electron pairs include, but are not limited to —CO₂H, —NO₂, amino, hydroxyl, thio, thioalkyl, —B(OH)₂, —SO₃H, PO₃H, phosphonate and heteroatoms in heterocycles. In some embodiments, the term “ligand” as described herein can refer to a group having two or more thio groups.

By “luminescence” is meant the emission of light.

By “macroscopic formulation” is meant a formulation comprising micelles, nanoparticles or polymers.

By “metal” is meant any of a class of elements which are generally electropositive including the d-block of the periodic table, the alkali and alkaline earth, as well as the heavier main-group or p-block elements.

By “NIR” is meant near infrared, commonly 700-1700 nm.

By “nitro” is meant an —NO₂ group.

By “pharmaceutically acceptable salt” is meant those salts which are, within the acceptance of sound medical judgment, suitable for use in contact with the tissues of humans and lower animals without undue toxicity, irritation, allergic response and the like, and are commensurate with a reasonable risk/benefit ratio.

By “phosphate” is meant a group derived from phosphoric acid. One example of phosphate includes a —O—P(═O)(OR^{P1})(OR^{P2}) or —O—[P(═O)(OR^{P1})—O]_{P3}—R^{P2} group, where each of R^{P1} and R^{P2}, is, independently, H, optionally substituted alkyl, optionally substituted alkenyl, optionally substituted aryl, or optionally substituted arylalkylene, and where P3 is an integer from 1 to 5. Yet other examples of phosphate include orthophosphoric acid, pyrophosphoric acid, tripolyphosphoric acid, tetrapolyphosphoric acid, trimetaphosphoric acid, and/or phosphoric anhydride, or combinations thereof.

By “phosphono” or “phosphonic acid” is meant a —P(O)(OH)₂ group.

By “spirocyclyl” is meant an alkylene diradical, both ends of which are bonded to the same carbon atom of the parent group to form a spirocyclyl group and also a heteroalkylene diradical, both ends of which are bonded to the same atom. Non-limiting alkylene and heteroalkylene groups for use within a spirocyclyl group includes C₂₋₁₂, C₂₋₁₁, C₂₋₁₀, C₂₋₉, C₂₋₈, C₂₋₇, C₂₋₆, C₂₋₅, C₂₋₄, or C₂₋₃ alkylene groups, as well as C₁₋₁₂, C₁₋₁₁, C₁₋₁₀, C₁₋₉, C₁₋₈, C₁₋₇, C₁₋₆, C₁₋₅, C₁₋₄, C₁₋₃, or C₁₋₂ heteroalkylene groups having one or more heteroatoms.

By “sulfate” is meant a group derived from sulfuric acid. One example of sulfate includes a —O—S(═O)₂ (OR^{S1}) group, where R^{S1} is H, optionally substituted alkyl, optionally substituted alkenyl, optionally substituted aryl, or optionally substituted arylalkylene. By “sulfo” or “sulfonic acid” is meant an —S(O)₂OH group.

By “sulfonyl” is meant an —S(O)₂— or —S(O)₂R group, in which R can be H, optionally substituted alkyl, or optionally substituted aryl. Non-limiting sulfonyl groups can include a trifluoromethylsulfonyl group (—SO₂—CF₃ or Tf).

By “thiocyanato” is meant an —SCN group.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S M et al., “Pharmaceutical salts,” J. Pharm. Sci. 1977 January; 66 (1): 1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, glucoheptonate, gluconate, glutamate, glycerophosphate, hemisulfate, heptonate, hydrobromide, hexanoate, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine. Yet other salts include ammonium, sulfonium, sulfoxonium, phosphonium, iminium, imidazolium, benzimidazolium, amidinium, guanidinium, phosphazinium, phosphazenium, pyridinium, etc., as well as other cationic groups described herein (e.g., optionally substituted isoxazolium, optionally substituted oxazolium, optionally substituted thiazolium, optionally substituted pyrrolium, optionally substituted furanium, optionally substituted thiophenium, optionally substituted imidazolium, optionally substituted pyrazolium, optionally substituted isothiazolium, optionally substituted triazolium, optionally substituted tetrazolium, optionally substituted furazanium, optionally substituted pyridinium, optionally substituted pyrimidinium, optionally substituted pyrazinium, optionally substituted triazinium, optionally substituted tetrazinium, optionally substituted pyridazinium, optionally substituted oxazinium, optionally substituted pyrrolidinium, optionally substituted pyrazolidinium, optionally substituted imidazolinium, optionally substituted isoxazolidinium, optionally substituted oxazolidinium, optionally substituted piperazinium, optionally substituted piperidinium, optionally substituted morpholinium, optionally substituted azepanium, optionally substituted azepinium, optionally substituted indolium, optionally substituted isoindolium, optionally substituted indolizinium, optionally substituted indazolium, optionally substituted benzimidazolium, optionally substituted isoquinolinum, optionally substituted quinolizinium, optionally substituted dehydroquinolizinium, optionally substituted quinolinium, optionally substituted isoindolinium, optionally substituted benzimidazolinium, and optionally substituted purinium). Yet other salts can include an anion, such as a halide (e.g., F⁻, Cl⁻, Br⁻, or I⁻), a hydroxide (e.g., OH), a borate (e.g., tetrafluoroborate (BF₄), a carbonate (e.g., CO₃²⁻ or HCO₃⁻), or a sulfate (e.g., SO₄²⁻). Cations can include, but are not limited to, alkali cations, phosphonium cations and tetraalkylammonium cations.

By “leaving group” is meant an atom (or a group of atoms) with electron withdrawing ability that can be displaced as a stable species, taking with it the bonding electrons, or an atom (or a group of atoms) that can be replaced by a substitution reaction. Examples of suitable leaving groups include H, halides, and sulfonates including, but not limited to, triflate (—OTf), mesylate (—OMs), tosylate (—OTs), brosylate (—OBs), acetate, Cl, Br, and I.

By “attaching,” “attachment,” or related word forms is meant any covalent or non-covalent bonding interaction between two components. Non-covalent bonding interactions include, without limitation, hydrogen bonding, ionic interactions, halogen bonding, electrostatic interactions, a bond interactions, hydrophobic interactions, inclusion complexes, clathration, van der Waals interactions, metallophilic interactions and combinations thereof.

### TTFtt Diradical Dication Imaging Applications

The TTFtt diradical dications emit brightly in the near infrared region (1000-1700 nm). NIR II luminescence is rare given the high probability of nonradiative decay at lower energy transitions; however, NIR II emitters are incredibly valuable for biomedical imaging applications as NIR II light falls in the tissue-transparent region. Since the NIR emission of the TTFtt diradical dications is sensitive to the redox state of the TTF core, exposure to different potentials enables redox-switchable photoluminescence, which can be used for biological sensing.

In one embodiment, the TTFtt diradical dications may have utility in the field of cancer, assisting with interoperative visualization of tumors to be removed. The present chromophoric compositions of Formula (I) could be conjugated at R₁ and/or R₂ positions to a tumor-specific targeting ligand that causes attachment of the chromophore to accumulate in cancers that over-express the ligand's receptor. Examples of tumor-targeting ligands include folic acid (which exhibits specificity for folate receptor positive cancers of the ovary, kidney, lung, endometrium, breast and colon) and (2S,2'S)-2,2′-(carbonyldiimino)dipentanedioic acid (DUPA) which can deliver attached fluorophores selectively to cells expressing prostate-specific membrane antigens such as prostate cancers.

### TTFtt Diradical Dication as Optically Addressable Qubits

The dications possess diradical character that forms a two-state system called a qubit, or quantum bit. This qubit can be manipulated with microwaves with a long room temperature lifetime. TTFtt diradical dications are thus optically addressable qubit candidates. Optically addressable qubits allow for initialization and readout of spin using light. They are orders of magnitude more sensitive than the leading magnetometers and can detect incredibly weak magnetic fields at relatively long distances.

In some embodiments, the coordination complexes are advantageously used as qubits (i.e. quantum bits), qutris and other types of quantum resources. These embodiments include methods for preparing and utilizing coordination complexes for quantum computation and information processing, quantum communication and teleportation, quantum memories, sensing and other quantum-mechanical applications. Accordingly, the coordination complexes may also be referred to as “molecular-spin qubits”. For example, some of the methods presented herein can be used to initialize a coordination complex via spin polarization. Other methods can be used to coherently control a spin-polarized coordination complex to deterministically place the coordination complex in a specific quantum state. At the end of a quantum computation or sensing sequence, additional methods can be used to determine the spin population of the coordination complex by measuring photoluminescence (i.e.—resonance phosphorescence) emitted by the coordination complex during optical pumping.

An aspect is that the electronic structure of an atom can be modified in numerous ways due to its interaction with ligands, therefore allowing the electronic structure to be “chemically tuned” by selection of particular ligands. This ability to chemically tune atomic structure advantageously gives rise to a significantly greater variety of electronic structures as compared to that of the bare atom. Such variety increases the likelihood of finding electronic structures that are particularly useful for implementing the TTFtt molecules with existing technologies. For example, some of the modified electronic structures have transitions that can be tuned to coincide with readily-available laser lines and microwave sources.

In some embodiments, the TTFtt diradical dications' qubit characteristics can be leveraged for cellular sensing or imaging applications.

### TTFtt Diradical Dication Magnetic Resonance Imaging Applications

Magnetic Resonance Imaging (MRI) is an imaging technique which is not dependent on tissue depth and does not require radioisotopes. It is a diagnostic technique that allows imaging of optically opaque subjects and provides contrast among soft tissues at high spatial resolution. Gadolinium and magnetite nanoparticles have been used as contrast-enhancing agents for MRI.

In the majority of clinical applications, an MRI signal is derived from protons of the water molecules present in the materials being imaged. The image intensity of tissues is determined by a number of factors. The physical properties of a specific tissue, such as the proton density, spin lattice relaxation time (T1), and the spin-spin relation time (T2) often determine the amount of signal available. Depending on the properties of the contrast agents, the TI (longitudinal) or T2 (transverse) weighted images or both may be altered. Methods to increase the resolution of MRI imaging include: extending the scan time, using high efficiency coils, increasing field strength and increasing the accumulation of contrast agent in cells or tissues.

A number of compositions termed “contrast agents” have been developed to provide enhanced contrast between different tissues. Contrast agents commonly affect T1, T2 or both. In general, contrast agents are made potent by incorporating metals with unpaired d or f electrons. For example, T1 contrast agents often include a lanthanide metal ion, usually Gd³⁺, that is chelated to a low molecular weight molecule in order to limit toxicity. T2-agents often consist of small particles of magnetite (FeO—Fe₂O₃) that are coated with dextran. Both types of agents interact with mobile water in tissue to produce contrast. The details of the microscopic interaction differ depending upon the type of agent used.

The tetrathiafulvalene-2,3,6,7 tetrathiolate bridged bimetallic complexes of the present disclosure may be particularly advantageous contrast agents since there is flexibility in how they may be activated and then detected. Near infrared (NIR) absorbance of these complexes optically polarizes their nuclear spins for imaging. Any nuclear spin on the complex could be polarized through dynamic nuclear polarization, by optically exciting the electronic NIR transition which then could transfer polarization to the nuclei. Given a simple, all-optical pulse sequence, the data shows that it is possible to polarize the lower triplet state, giving a PL contrast of 16% in acetonitrile. Separately, by exciting the complexes with NIR light, polarization may be transferred to surrounding water molecules, in turn enabling higher relaxivity. Therefore, the complexes directly may serve as optically gated contrast agents. The redox state of these complexes can also be advantageously manipulated for their use as redox-responsive sensors. The complexes are useful for both in vitro and in vivo imaging.

When the imaging is in vivo, the preferred administration route for the contrast agent is parenteral, such as by bolus injection, by intravenous, intra-arterial or peroneal injection. The lungs may be imaged by spray such as an aerosol spray. Parenteral compositions may be injected directly or mixed with a large volume parenteral composition for systemic administration. Formulations for the enteral administration may vary widely. In general, such formulations include a diagnostically effective amount of the tetrathiafulvalene-2,3,6,7 tetrathiolate bridged bimetallic complex. Such enteral compositions may optionally include buffers, surfactants, or thixotropic agents among others. Compositions for oral administration may also contain flavoring agents and other ingredients for enhancing their organoleptic qualities.

The contrast agents are administered in a dose effective to achieve the desired enhancement of the MRI image. Such doses may vary widely, depending upon several factors including the target which is the subject of the imaging procedure, and the MRI imaging equipment utilized. The contrast agents are used in a conventional manner in MRI procedures. The contrast agents may be administered in a sufficient amount to provide adequate visualization; to a warm-blooded mammal either systematically or locally to an organ or tissue to be imaged, and the mammal subjected to the MRI procedure.

### TTFtt Diradical Dication Theranostics Applications

In some embodiments, the present dication tetrathiafulvalene-2,3,6,7-tetrathiolate bridged bimetallic compounds with diradical character may be therapeutic agents themselves. In some embodiments the present dication tetrathiafulvalene-2,3,6,7-tetrathiolate bridged bimetallic compounds with diradical character may be attached to a moiety with therapeutic properties.

In some embodiments, the present dication tetrathiafulvalene-2,3,6,7-tetrathiolate bridged bimetallic compounds with diradical character can simultaneously serve as delivery vehicles for therapeutic agents and as luminescent agents. Such theranostics may be based upon compounds of formula (II) wherein a therapeutic agent can be a substituent at any R₃ or R₄ position. For example, the therapeutic agent can be directly appended to the metal or attached to the metal by a linking moiety as shown in Formula (III), or via attachment to R₃ and R₄ to form a ring as shown in Formula (IV). The therapeutic agent could also be attached to the TTFtt bridge as a substituent of a ring formed from R₃ and R₄ as shown in Formula (V).

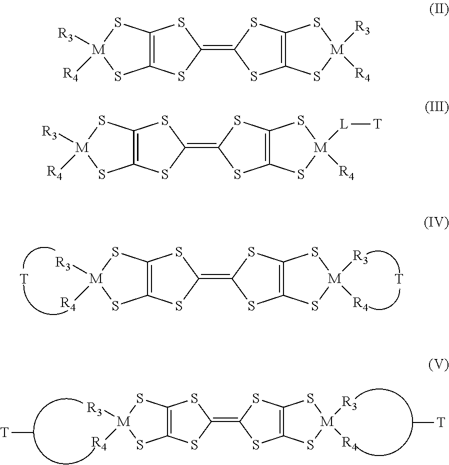

The therapeutic agents include analgesics, anaesthetics, antibacterials, anticonvulsants, antifungals, anti-inflammatoires, antineoplastics or anti-parasitics.

The linking moiety can be between the TTFtt structure and another moiety (e.g., the therapeutic agent) or between two (or more) other moieties. Linking moieties (e.g., L, L¹, L², L³, L⁴, L^{A}, L^{A′}, L^{A″}, L^{B′}, L^{B″}, L^{8A}, and others) can be any useful multivalent group, such as multivalent forms of optionally substituted aliphatic, optionally substituted heteroaliphatic, optionally substituted aromatic, or optionally substituted heteroaromatic.

Non-limiting linking moieties (e.g., L in Formula (III)) include a covalent bond, a spirocyclic bond, —O—, —NR^{N1}—, —C(O)—, —C(O)O—, —OC(O)—, —SO₂—, optionally substituted alkylene, optionally substituted alkyleneoxy, optionally substituted haloalkylene, optionally substituted heterocyclyl, optionally substituted heteroalkylene, optionally substituted arylene, optionally substituted aryleneoxy, optionally substituted heterocyclyldiyl, —SO₂—NR^{N1}-Ak-, —(O-Ak)_{L1}-SO₂—NR^{N1}-Ak-, -Ak-, -Ak-(O-Ak)_{L1}-, —(O-Ak)_{L1}-, -(Ak-O)_{L1}—, —C(O)O-Ak-, —Ar—, or —Ar—O—, as well as combinations thereof. In particular embodiments, Ak is an optionally substituted aliphatic, optionally substituted alkylene, or optionally substituted haloalkylene; R^{N1} is H or optionally substituted alkyl or optionally substituted aryl; Ar is an optionally substituted aromatic or optionally substituted arylene; and L1 is an integer from 1 to 3.

In some embodiments, the linking moiety is —(CH₂)_{L1}—, —O(CH₂)_{L1}—, —(CF₂)_{L1}—, —O(CF₂)_{L1}—, or —S(CF₂)_{L1}— in which L1 is an integer from 1 to 3. In other embodiments, the linking moiety is -Ak-O—Ar-Ak-O-Ak- or -Ak-O—Ar—, in which Ak is optionally substituted alkylene or optionally substituted haloalkylene, and Ar is an optionally substituted arylene. Non-limiting substituted for Ar includes-SO₂-Ph, in which Ph can be unsubstituted or substituted with one or more halo or pseudo-halo (azide) groups.

These compounds also have photothermal applications. Because some compounds have lower quantum yields and intense NIR absorptions (˜80,000 M⁻¹ cm⁻¹) along commonly used laser lines (808 nm and 1064 nm), they are strong photothermal agent candidates. The monocation monoradical complexes also have NIR absorptions that are non-emissive and can be used for these applications as well. This can lead to in situ ablation in biomedical settings.

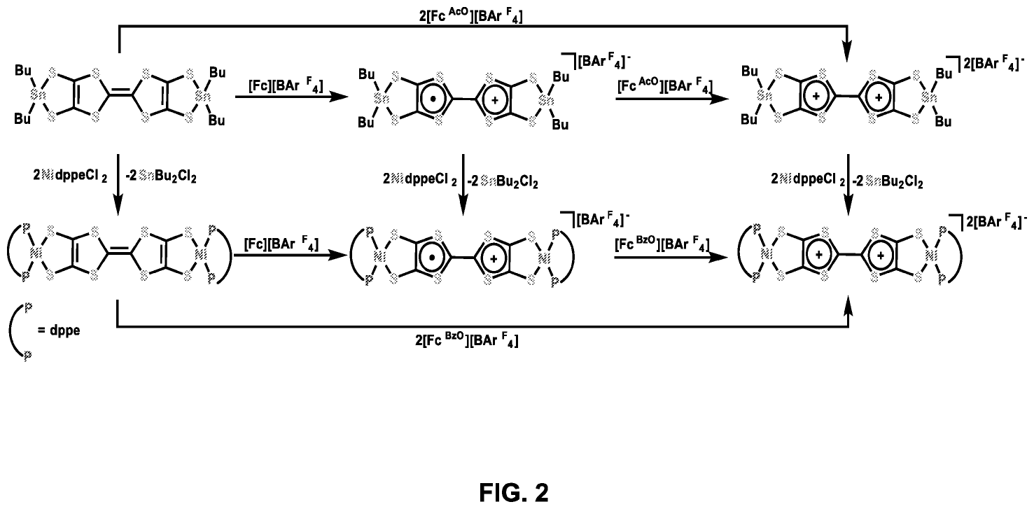

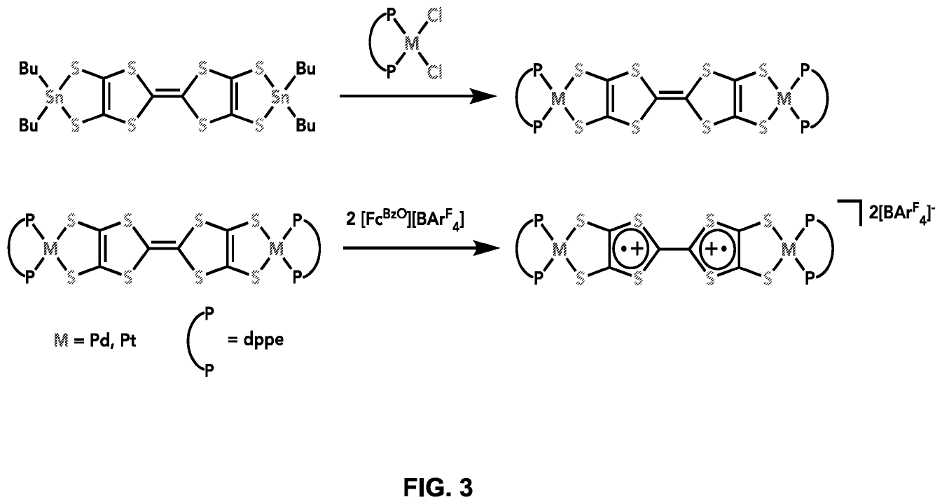

Synthesis of the compounds and their properties are described in detail hereinafter in the Examples. One synthetic route for the formation of the TTFtt coordination complexes via transmetalation is illustrated in FIGS. 2 and 3. The Examples have been included to provide guidance for practicing representative embodiments of the presently disclosed subject matter. In light of the present disclosure and the general level of skill in the art, it can be understood that the Examples are intended to be exemplary only and that numerous changes, modifications and alterations can be made without departing from the spirit and scope of the invention.

### General Methods

The complexes [(Pd{dppe})₂TTFtt][BAr^{F}₄]₂ (1), [(Pt{dppe})₂TTFtt][BAr^{F}₄]₂ (2), and [(Pt{P(4-CF₃Ph)₃}₂)₂TTFtt][BAr^{F}₄]₂ (3) were synthesized via a transmetallation between the appropriate phosphine supported dihalide precursors and the TTFtt synthon (SnBu₂)₂TTFtt followed by in-situ oxidation using two equivalents of [Fc^{BzO}][BAr^{F}₄] (where Fc^{BzO}=benzoylferrocenium, dppe=1,2-bis(diphenylphosphino) ethane and BAr^{F}₄=tetrakis(3,5-bis(trifluoromethyl)phenyl)borate. All three dicationic complexes were obtained as crystalline solids in high yields and single crystal X-ray diffraction (SXRD) of 1, 2, and 3 reveals that all three dications display planar P₂M₂TTFtt geometries with no π-stacking in the solid state. TTF units have a high propensity to T-stack, and we propose that shielding of the TTF core by the large BAr^{F}₄ anions prevents oligomerization. Such shielding is particularly apparent in the structure of 3 where fluorophilic interactions between the capping phosphine ligands and the [BAr^{F}₄]⁻ counter anions lead to encapsulation of the dicationic TTFtt core.

### Example 1

### Synthesis of [(Pd{dppe})₂TTFtt][BAr^{F}₄]₂

TTFtt (SnBu₂)₂ (0.017 g, 0.0217 mmol) was dissolved in 3 mL of DCM and slowly added to a suspension of Pd (dppe)₂Cl₂ (0.025 g, 0.0434 mmol) in 3 mL of DCM. The light yellow suspension formed a shiny dark pink suspension upon addition of the orange solution of TTFtt (SnBu₂)₂, and was left to stir for 10 mins. Next, [Fc^{BzO}][BAr^{F}₄] (0.053 g, 0.0456 mmol) was dissolved in 2 mL DCM and slowly added to the stirring reaction. The dark pink suspension turned olive green upon addition of [Fc^{BzO}][BAr^{F}₄] and was left to stir 5 mins. The solution was condensed to 1 mL under vacuum and petroleum ether (5 mL) was slowly added to further precipitate out the final product. The solids were washed with petroleum ether several times (3×5 mL) and dried under vacuum. The crude product was redissolved in 1 mL of DCM, filtered through celite, and layered with petroleum ether. The layered solution was cooled to −35° C. and left to crystallize overnight. The crystals were collected and dried (0.051 g, 76.9% yield). Crystals suitable for SXRD were selected from a 1,2-dichloroethane/petroleum ether slow diffusion gradient left to crystallize over a 2 day period at −35° C. ¹H NMR (400 MHZ, CD₂Cl₂, 298 K): δ 2.69 (d, dppe), 7.55 (s, [BAr^{F}₄]), 7.56-7.58 (m, dppe), 7.63-7.69 (m, dppe), 7.72 (s, [BAr^{F}₄]). ³¹P{¹H} NMR (162 MHZ, CD₂Cl₂, 298 K): δ 62.00. Anal. calc. for 1, C₁₂₂H₇₂B₂F₄₈Pd₂P₄S₈: C, 47.81%, H, 2.37%, N, 0%; found: C, 47.45%, H, 2.49%, N, none.

### Example 2

### Synthesis of [(Pt{dppe})₂TTFtt][BAr^{F}₄]₂

TTFtt (SnBu₂)₂ (0.042 g, 0.0527 mmol) was dissolved in 3 mL of DCM and slowly added to a suspension of Pt (dppe)₂Cl₂ (0.070 g, 0.1054 mmol) in 4 mL of DCM. The white suspension formed a shiny pink suspension upon addition of TTFtt (SnBu₂)₂, and was left to stir for 10 mins. Next, [Fc^{BzO}][BAr^{F}₄] (0.122 g, 0.1054 mmol) was dissolved in 4 mL DCM and slowly added to the stirring reaction. The shiny pink suspension turned olive green upon addition of [Fc^{BzO}][BAr^{F}₄] and was left to stir for 5 mins. The solution was condensed to 1 mL under vacuum and petroleum ether (5 mL) was slowly added to further precipitate out the final product. The solids were washed with petroleum ether several times (3×5 mL) and dried under vacuum. The crude product was redissolved in 1 mL of DCM, filtered through celite, and layered with petroleum ether. The layered solution was cooled to −35° C. and left to crystallize overnight. The crystals were collected and dried (0.152 g, 88.9% yield). Crystals suitable for SXRD were selected from a 1,2-dichloroethane/petroleum ether slow diffusion gradient left to crystallize over a 2 day period at −35° C. ¹H NMR (400 MHZ, CD₂Cl₂, 298 K): δ 2.62 (d, dppe), 7.55 (s, [BAr^{F}₄]), 7.57-7.62 (m, dppe), 7.64-7.70 (m, dppe), 7.72 (s, [BAr^{F}₄]). ³¹P{¹H} NMR (162 MHZ, CD₂Cl₂, 298 K): δ 47.09 (J_{Pt,P}=2919 Hz). ¹⁹⁵Pt{1H} NMR (107 MHZ, CD₂Cl₂, 298 K): δ −4597.51. Anal. calc. for 2, C₁₂₂H₇₂B₂F₄₈Pt₂P₄S₈: C, 45.19%, H, 2.24%, N, 0%; found: C, 45.31%, H, 2.59%, N, none.

### Example 3

### Synthesis of [(Pt{P(4-CF₃Ph)₃}₂)₂TTFtt][BAr^{F}₄]₂

TTFtt (SnBu₂)₂ (0.023 g, 0.0292 mmol) was dissolved in 3 mL of DCM and slowly added to a solution of Pt (P(4-CF₃Ph) 3)₂Cl₂ (0.070 g, 0.0584 mmol) in 3 mL of DCM. The colorless solution turned dark brown upon addition of TTFtt (SnBu₂)₂, and was left to stir for 5 mins. Next, [Fc^{BzO}][BAr^{F}₄] (0.067 g, 0.0584 mmol) was dissolved in 2 mL DCM and slowly added to the stirring reaction. The dark brown solution turned green upon addition of [Fc^{BzO}][BAr^{F}₄] and began to crystallize out of solution. The solution was condensed to 1 mL under vacuum and petroleum ether (5 mL) was slowly added to further precipitate out the final product. The solids were washed with petroleum ether several times (3×5 mL) and dried under vacuum. The crude product was redissolved in 1 mL of DCM, filtered through celite, and layered with petroleum ether. The layered solution was cooled to −35° C. and left to crystallize overnight. The crystals were collected and dried (0.089 g, 70.7% yield). Crystals suitable for SXRD were selected from a 1,2-dichloroethane/petroleum ether slow diffusion gradient left to crystallize over a 2 day period at −35° C. ¹H NMR (400 MHZ, CD₂Cl₂, 298 K): δ 7.48 (s, [BAr^{F}₄]), 7.51 (d, PhCF₃), 7.62 (d, PhCF₃), 7.70 (s, [BAr^{F}₄]). ³¹P{¹H} NMR (202 MHZ, CD₂Cl₂, 298 K): § 14.92 (J_{Pt,P}=2992 Hz). ¹⁹⁵Pt{1H} NMR (107 MHz, CD₂Cl₂, 298 K): δ −4490.87. Anal. calc. for 3, C₁₅₄H₇₂B₂F₈₄Pt₂P₄S₈: C, 42.91%, H, 1.69%, N, 0%; found: C, 42.36%, H, 1.87%, N, none.

### Example 4

### UV-Vis-NIR Property Analysis

UV-Vis-NIR measurements were performed using a Shimadzu UV-3600 Plus dual beam spectrophotometer. UV-Vis region spectra were acquired on a Thermo Scientific Evolution 300 spectrometer with the VISIONpro software suite. Variable temperature measurements were performed using an Unisoku CoolSpeK 203-B cryostat.

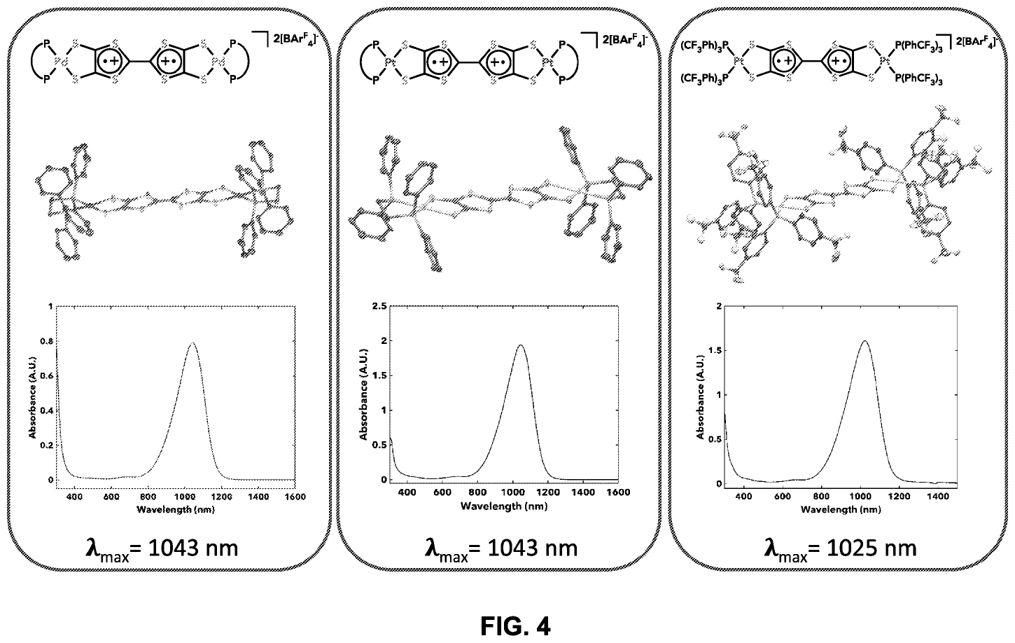

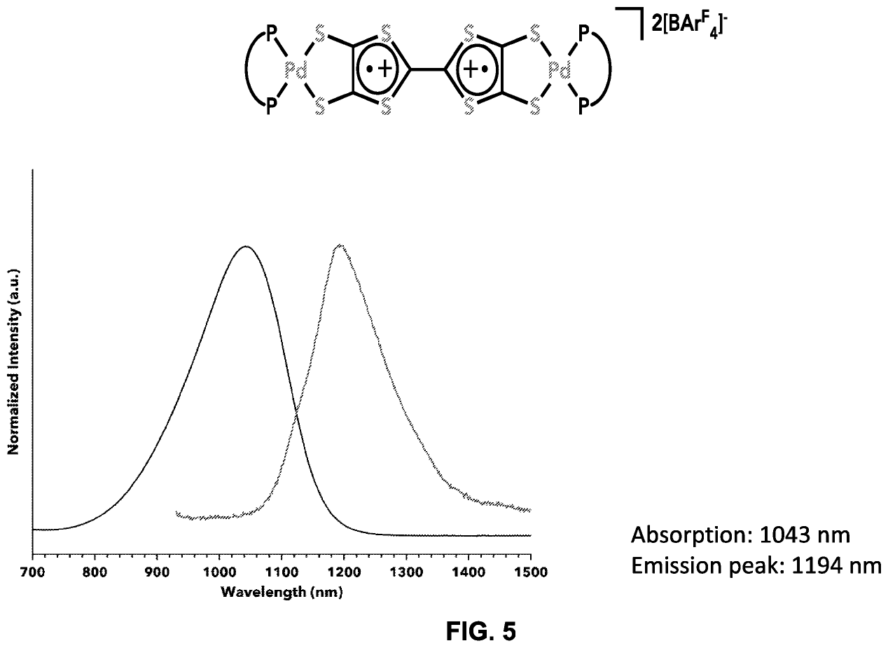

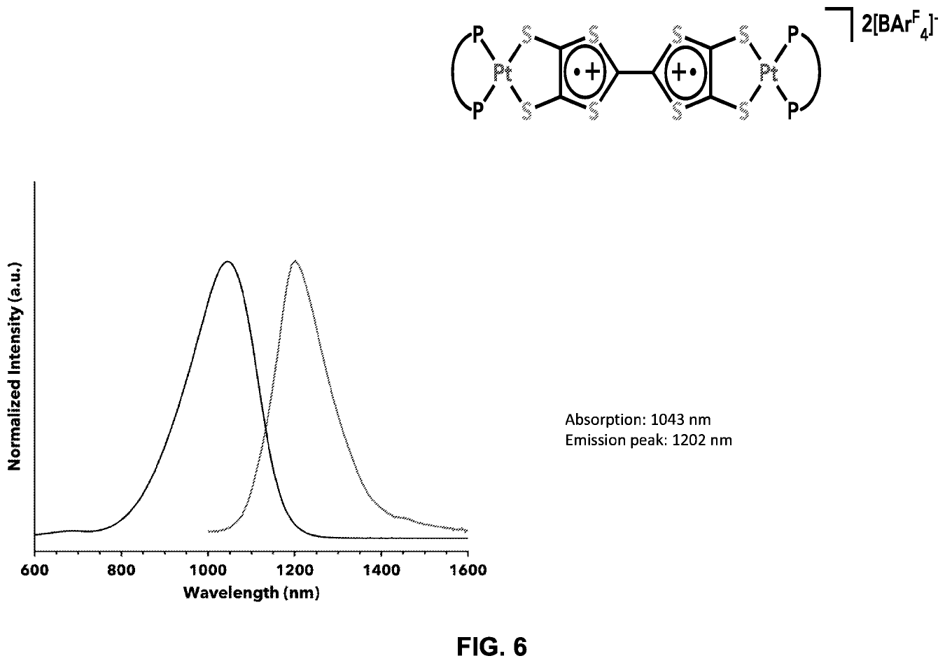

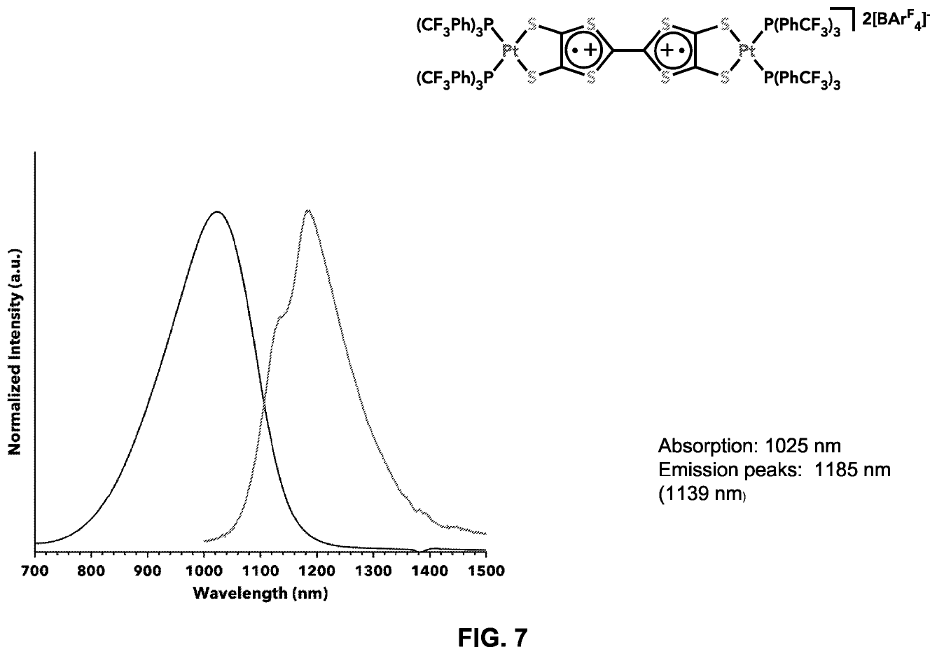

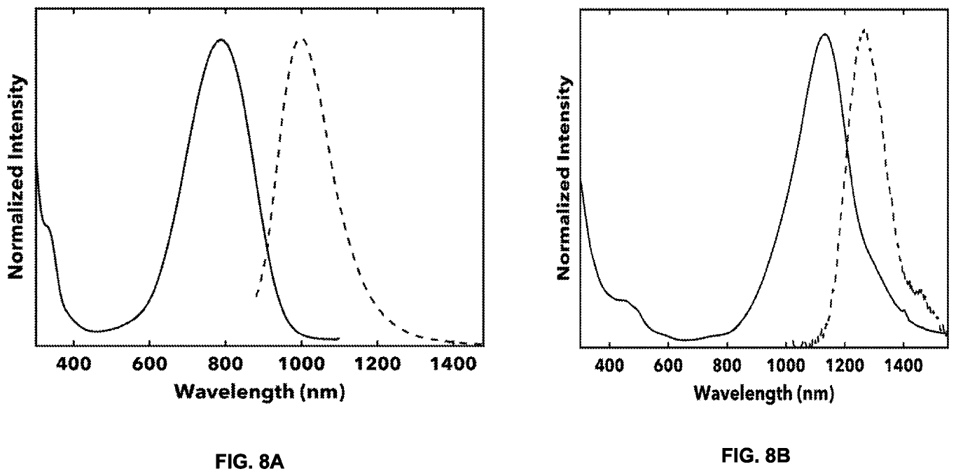

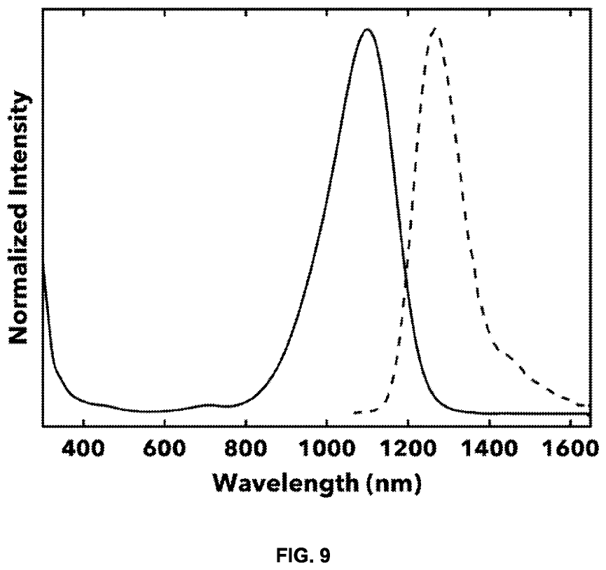

Measurements revealed that compounds 1-3 all have intense NIR absorbances at ˜1000 nm (as illustrated in FIG. 4) which can be assigned to low energy π-π transitions. Upon NIR excitation (900 nm) all three complexes exhibit intense emission centered at ˜1200 nm (as illustrated in FIGS. 5-7). A minimal hypsochromic shift is apparent in the NIR absorption maximum and photoluminescence (PL) spectra of compound 3; however, at 77 K, its PL spectrum aligns with the other analogues. The strong NIR absorptions are remarkably tunable, and can shift from 788 nm to 1134 nm, dependent on solvent and metal/capping ligand combination (FIGS. 8A-8B). These absorptions cover both the NIR I and II ranges, allowing for flexibility of irradiation source choice. Similarly, PL can also shift with solvent and metal/capping ligand combination, from 998 nm to 1267 nm, with tails trailing out to 1600 nm (FIG. 9).

### Example 5

### Photoluminescence Quantum Yield Determination

For the photoluminescence spectroscopy, all emission spectra were acquired on a Horiba Scientific PTI QuantaMaster fluorometer. Low temperature emission spectra were recorded by adding samples in 1:1 DCM:toluene into quartz EPR tubes and lowering the tube into an optical dewar filled with liquid N₂. Excitation emission matrices were acquired on a Horiba Scientific Fluorolog-3 spectrofluorometer.

The samples, sealed in NMR tubes, were individually placed in an integrating sphere (Thorlabs, IS200-4) with a PbSe detector (Thorlabs, PDA20H) on one port and directly excited by an 808 nm diode laser (Thorlabs, M9-808-0150) of 15 W/cm² average intensity under 1 kHz square-wave modulation. The detector signal S, with and without a silicon window on the detector port, was recorded with the sample and with a DCM blank. 808 nm light is completely blocked by the silicon window within the sensitivity of the measurement. The photoluminescence quantum yield is therefore S_{S,Si}/(S_{D}−S_{S})×( [symbol] ₈₀₈/ [symbol] _{λ}_{_{S}})×T⁻¹ where the subscripts S, D and Si respectively denote the sample, DCM blank, and silicon window. R is the wavelength-dependent PbSe detector responsivity, T is the total emission power transmitted by the silicon window, and λ_{S} is the average emission wavelength determined by integration of the spectrum. The detector responsivity is linear with wavelength in this spectral range so that [symbol] ₈₀₈/ [symbol] _{λ}_{_{S}}=808/λ_{S}. Due to the spectral overlap between the sample emission and the soft absorption edge of silicon, we calculate T explicitly as ∫E(λ)dλ×

$${( \int \frac{E ( λ )}{T ( λ )} d λ )}^{- 1}$$

where E(λ) is the normalized sample emission spectrum and T(λ) is the T(λ) normalized silicon window transmission spectrum.

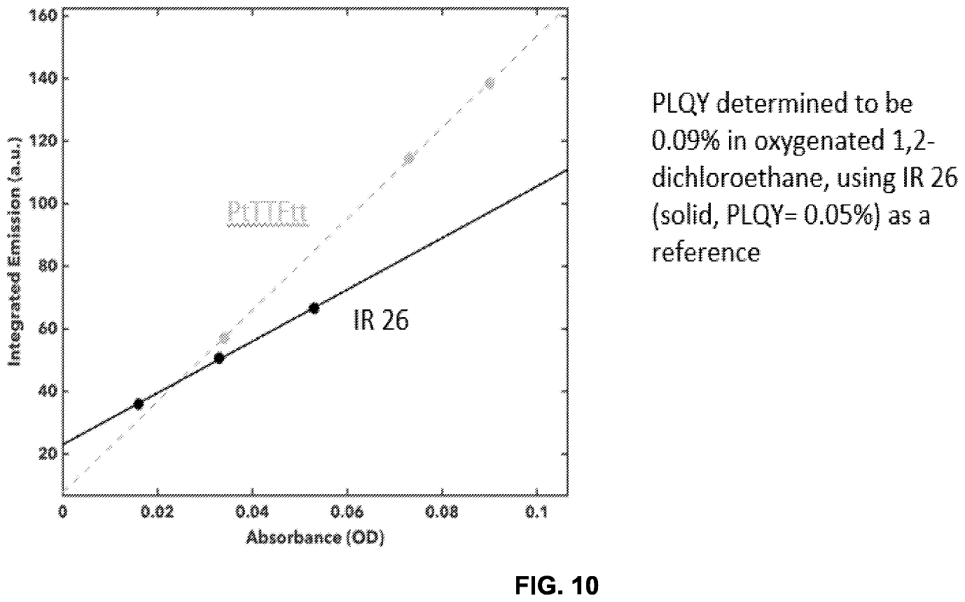

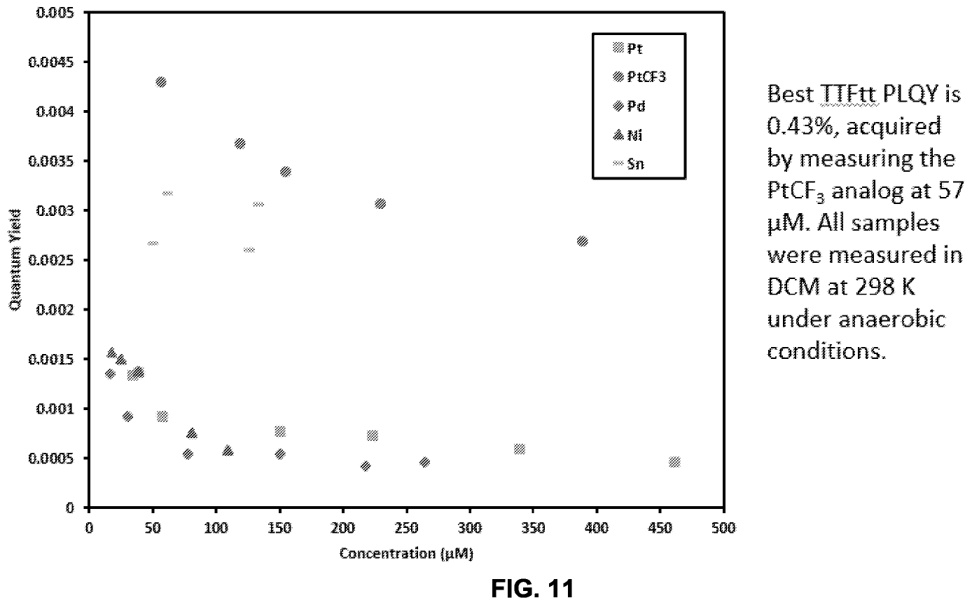

Comparative results for the diradical dication PtTTFtt against a conventional near IR dye (IR 26) provide PLQY of 0.09% in oxygenated 1,2-dichloroethane for compound 2 (PtTTFtt) in comparison to 0.05% for IR 26, as illustrated in FIG. 10. Testing of compounds 1-3 revealed that compound 3 provided the highest PLQY at 0.43% at 57 μM at 298K under anaerobic conditions as illustrated in FIG. 11.

### Example 6

### Cyclic Voltammetry of Compound 2

Cyclic voltammetry measurements were performed using a glassy carbon working electrode, a silver wire pseudoreference, and a platinum wire counter electrode. Each voltammogram was referenced to internal FcCp₂⁺/FcCp₂. All measurements were acquired using a BAS Epsilon potentiostat and analyzed using the BASi Epsilon software version 1.40.67NT.

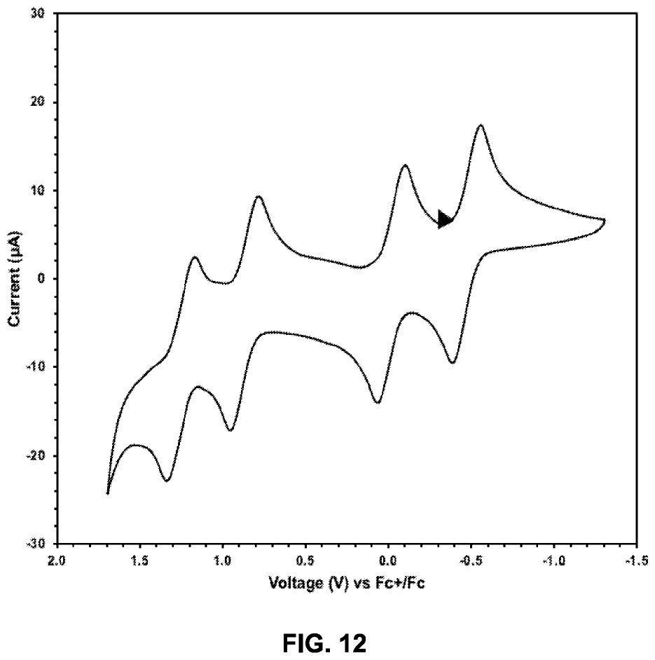

The redox states of the PtTTftt complex and their corresponding potentials are illustrated in the cyclic voltammogram of FIG. 12, for compound 2, PtdppeTTFtt, in dichloromethane (DCM). The two couples on the right (towards negative potentials) correspond to the TTFtt core, and the two couples on the left (towards positive potentials) are believed to correspond to metal-centered oxidation. This particular experiment was run using the monocationic form (as can be seen by location of arrow) and scanning right to reduce to the neutral complex and then scanning left to oxidize back to the monocation, dication, and then to the higher oxidation states (which have not yet been isolated). The spacing between the reduction and oxidation waves for each couple indicates that these features are roughly reversible, which is useful for the applications described herein.

### Example 7

### Electronic Structural Analysis

The diradical nature of compounds 1-3 was determined in the following manner. Electron paramagnetic resonance (EPR) measurements were conducted on a Bruker Elexsys E500 spectrometer equipped with a Bruker Cold-Edge stinger and an Oxford ESR 900 X-band cryostat.

A strong signal at g=2 suggestive of an organic radical was present in the electron paramagnetic resonance (EPR) spectra of these complexes at both 15 K and 298 K (see SI).³⁷ Variable temperature measurements showed this signal near g=2 growing in intensity as temperature is increased, suggesting a singlet ground state with thermal population of a triplet excited state.

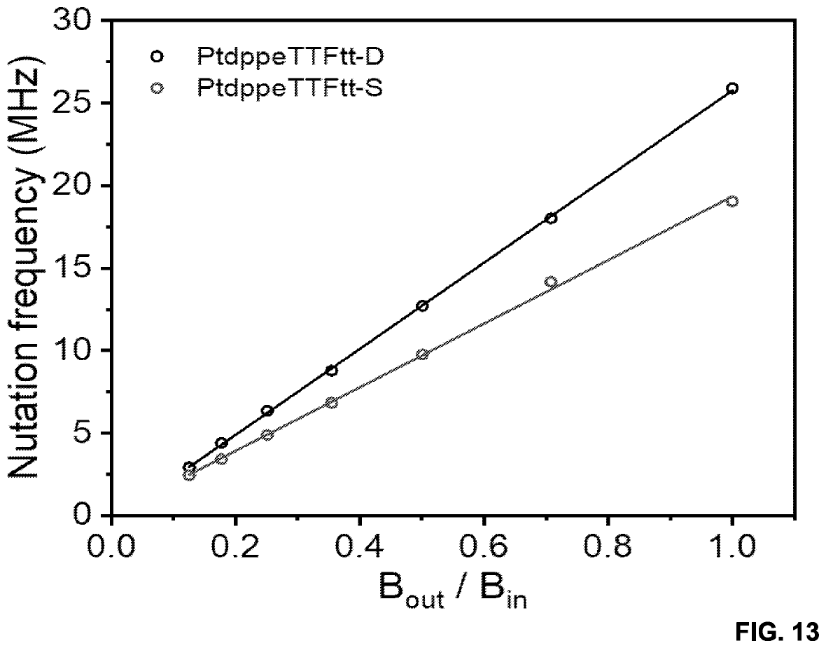

Furthermore, comparison with the PtdppeTTFtt monocation reveals a characteristic shift (sq[2]) of Rabi frequencies at a given microwave power, distinguishing the dication as a diradical species (FIG. 13). PtdppeTTFtt-D corresponding to the dication compound 2 is represented by the upper line in the graph, while PtddppeTTftt-S corresponding to the monoradical monocation is represented by the lower line in the graph.

These experimental findings were further supported by complete active space self-consistent field (CASSCF) calculations using the variational 2-RDM (V2RDM) method in the Maple Quantum Chemistry Package. An active space encompassing 20 electrons distributed in 20 spatial orbitals was used throughout the calculations and a 6-31G basis set was utilized for all atoms but Pd and Pt, for which the LANL2TZ basis set and effective core potential (ECP) was used.

The calculations revealed that all complexes exhibited singlet ground states with significant degrees of multireference correlation and biradical character. The triplet-singlet gaps obtained with V2RDM, as well as density functional theory (DFT) increase across the series of compounds 2<1<3 as the degree of fractional occupation in the HONO and LUNO orbitals decreases from λ_{LUNO,S}=0.46, λ_{HONO,S}=1.57 in compound 2 to λ_{LUNO,S}=0.33, λ_{HONO,S}=1.72 in 3. Inspection of the frontier NO densities reveals that the multireference correlation arises from near-degeneracy of the HONO and LUNO, which have A_{g} and Aᵤ symmetry and are π*-antibonding and π-bonding across the central TTFutt C—C bond, respectively.

### Example 8

### Study of Redox-Switchable Photoluminescence

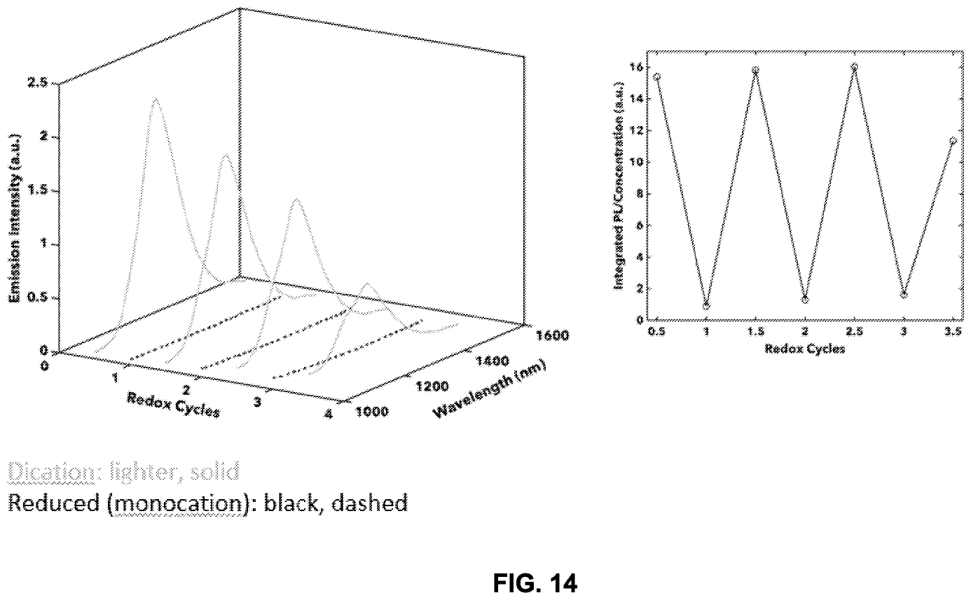

The redox-switchable photoluminesce of the compounds was studied in the following manner. Because of the reversible behavior illustrated by Example 7, a test was performed to determine if luminescence could be switched on and off upon the addition of oxidants and reductants. As shown by the graphs of FIG. 14, redox-switchable luminescence in DCM using PtdppeTTFtt, [Fc^{BzO}][BAr^{F}₄] (compound 2) as the oxidant, and CoCp₂ as the reductant was demonstrated. The UV-Vis (absorbance) and luminescence were measured before and after oxidation across three cycles. Each time, the luminescence was completely quenched upon reduction to the monocationic form, and subsequently recovered after re-oxidation to the dicationic complex. This switchable behavior could be advantageously leveraged for biological sensing applications. Furthermore, the monocationic and dicationic forms have different magnetic properties (monoradical vs. diradical), so these complexes are also promising magnetic/quantum sensing candidates.

### Example 9

### Study of Qubit Capability

The diradical character of the TTFtt compounds (as demonstrated in Example 7) forms a two-state system (qubit or quantum bit) which can be manipulated with microwaves.

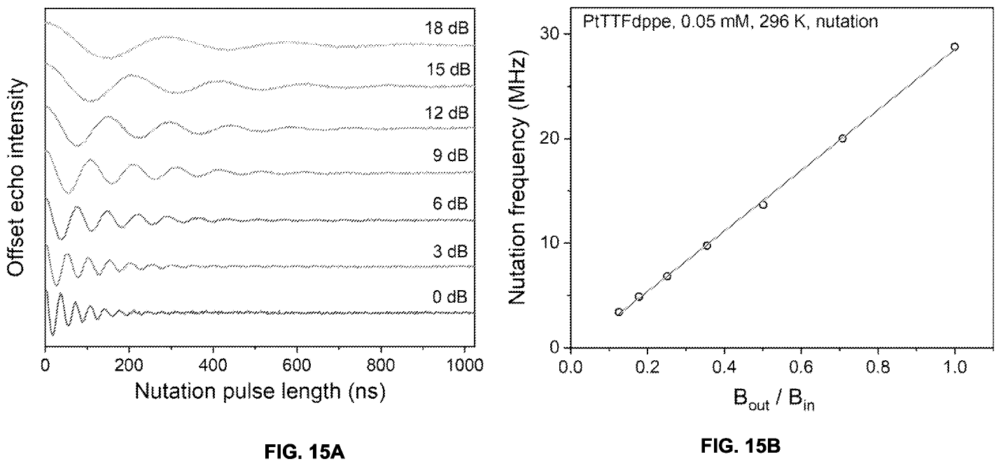

To test the viability of these systems as molecular qubits, the spins were manipulated with microwaves using pulsed electron paramagnetic resonance (EPR) sequences for PtdppeTTFtt, (compound 2). The attenuation (how much microwave power the sample sees) was changed across seven measurements as shown in FIG. 15A.

Next, the nutation frequency was plotted against the magnetic field as shown in FIG. 15B. This relationship is linear and passes through the origin, indicating that the nutation frequency can be used as a standard for magnetic field strength. The data shows that these spins can be coherently manipulated and detected, thus validating their use as qubits.

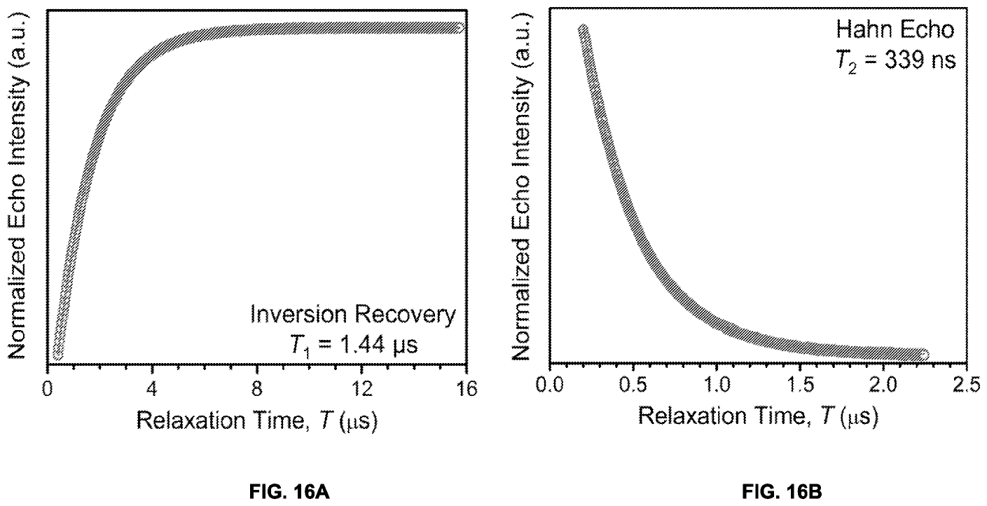

Furthermore, the spin lattice and spin dephasing times were measured for compound 2, PtdppeTTFtt, in DCM solution at room temperature (FIGS. 16A and 16B). The spin dephasing time falls within the operational regime (>0.2 μs) under these conditions, further supporting viability as a solution-phase, room temperature molecular qubit.

### Example 10

### Dication Diradical Character of Compound 4

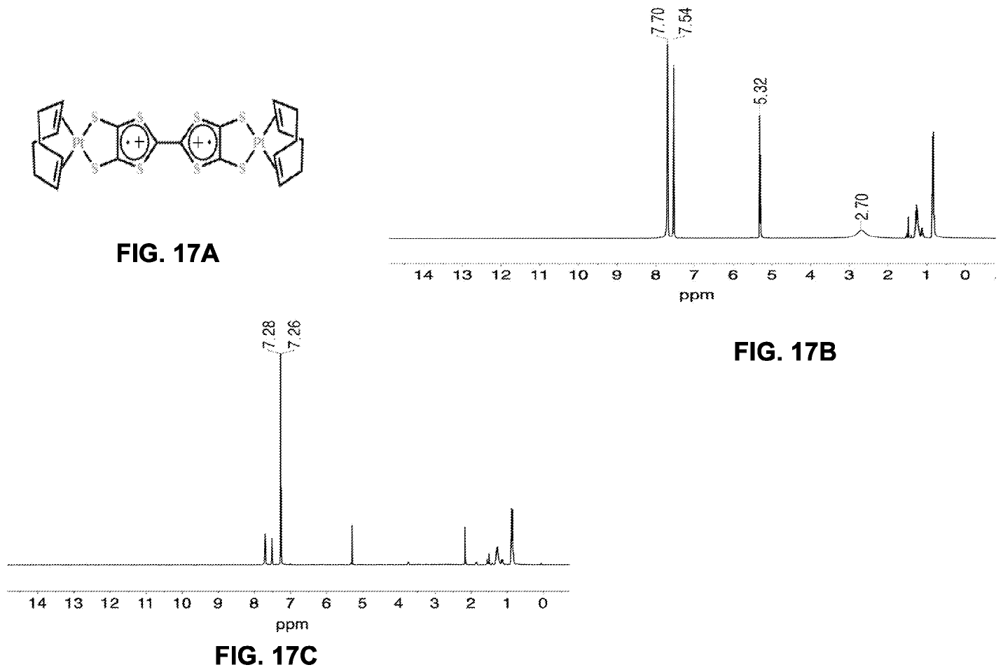

The structure of (Pt{1,5-cyclooctadiene})₂ tetrathiafulvalene-2,3,6,7 tetrathiolate diradical dication (compound 4 or PtCODTTFtt), synthesized according to the procedures described herein is shown in FIG. 17A. In compound 4, the TTFtt bridging structure forms a dimetallic Pt coordination complex with 1,5-cyclooctadiene.

Compound 4 was characterized with NMR spectroscopy. ¹H NMR spectra were acquired on Bruker DRX 400 and 500 spectrometers. Residual solvent peaks were referenced for all ¹H NMR measurements. FIG. 17B shows an ¹H NMR spectrum for compound 4. The broad peak at 2.70 ppm is indicative of diradical character localized on the TTFtt bridging structure.

Using the Evans method, paramagnetism of compound 4 was confirmed, as evidenced by a near perfect triplet magnetic moment of 2.79 in FIG. 17C. Evans method measurements were all conducted in CD₂Cl₂ with a capillary insert of 95/5 w/w % CD₂Cl/DCM. Pascal's constants were used to correct for the diamagnetic contribution of each complex.

### Example 11

### Photophysical Properties of Compound 4

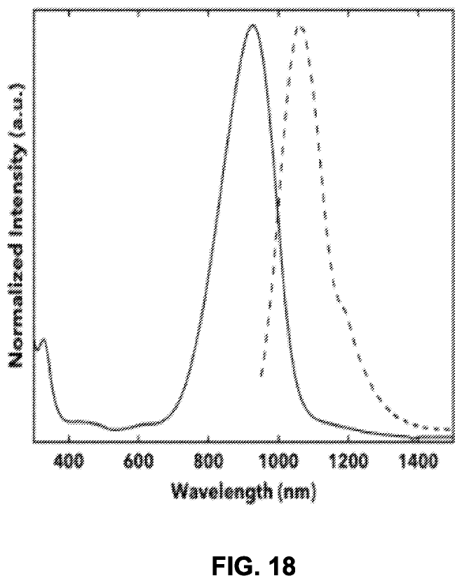

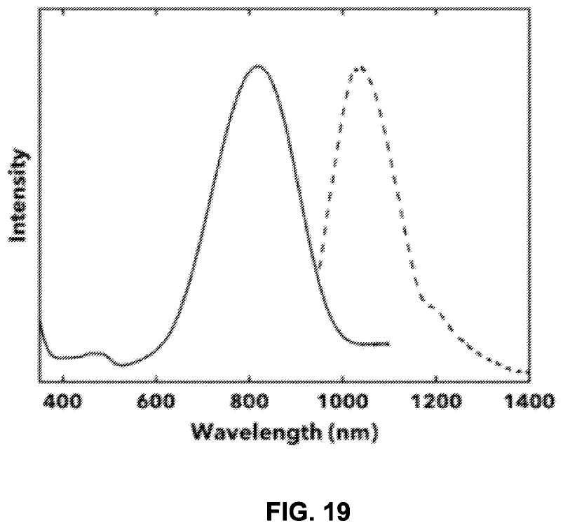

Following the procedure described in Example 5, the photophysical properties of compound 4 were explored. FIG. 18 demonstrates the blue shift of the absorption and emission maxima into a favorable region for laser and LED excitation and detection. The same trends in wavelength shift (FIG. 19) and PLQY are noted in acetonitrile solvent.

### Example 12

### Stability of Compound 4

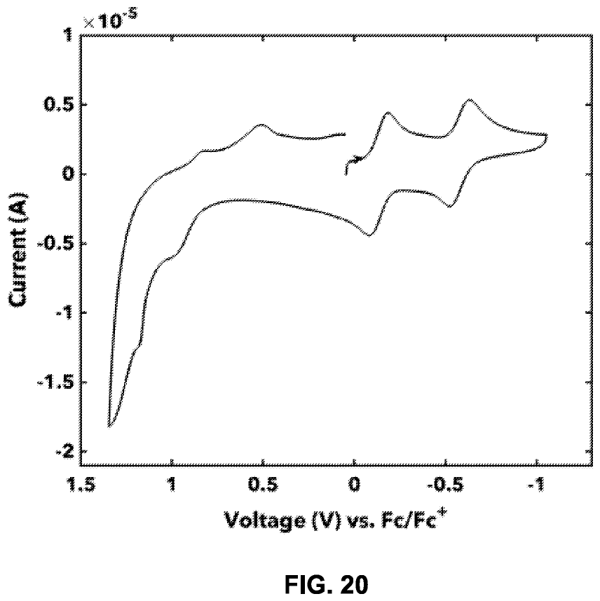

Compound 4 was also studied by cyclic voltammetry, in accordance with the procedure described in Example 6. FIG. 20 illustrates a cyclic voltammogram of compound 4, in dicloromethane, showing the TTFtt-centered redox waves are shifted negatively. The data suggests that PtCODTTFtt has increased stability. Based on the stability of compound 4, further testing demonstrated that photoluminesence persisted 21 hours with continuous irradiation. Stability to air, fetal bovine serum and phosphate buffer was also demonstrated.

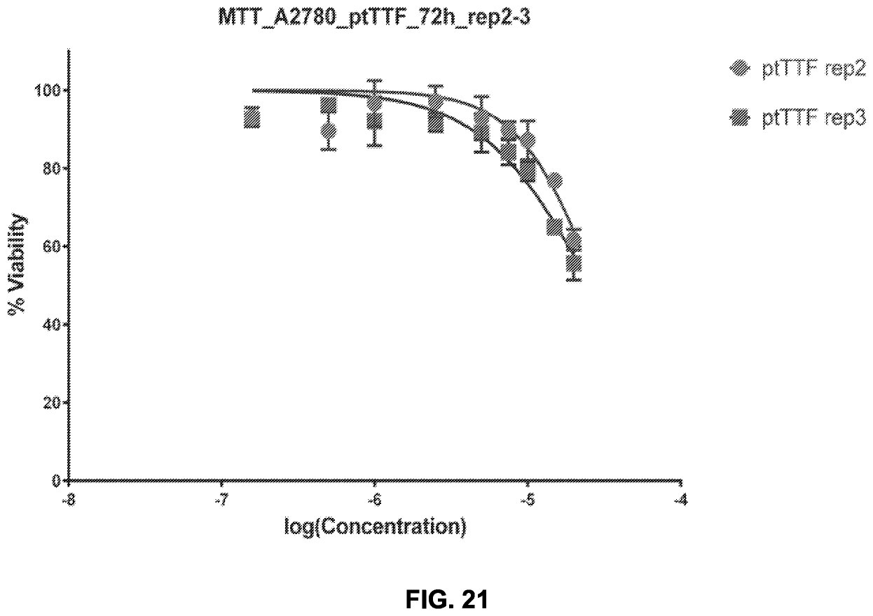

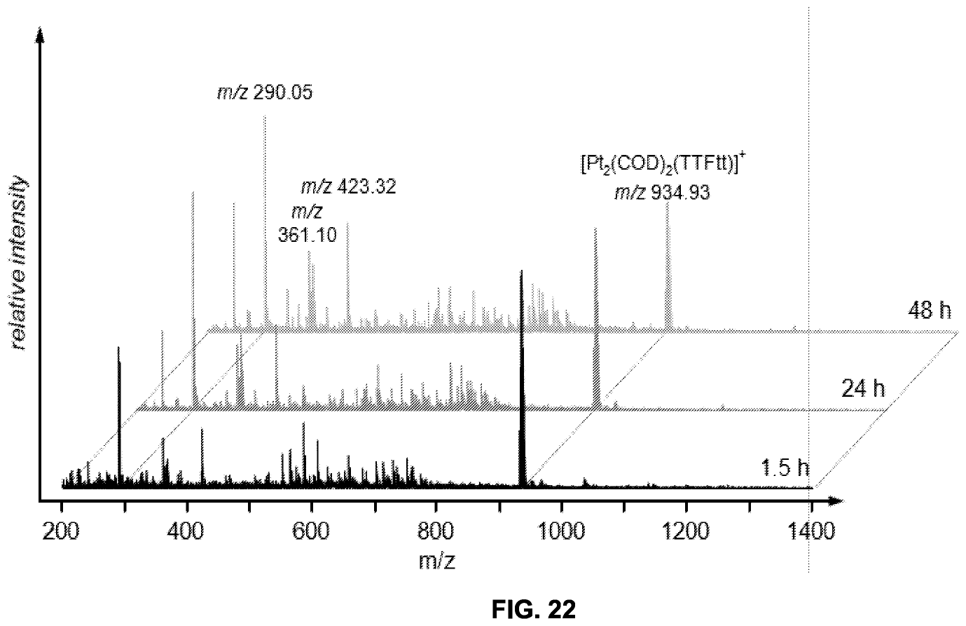

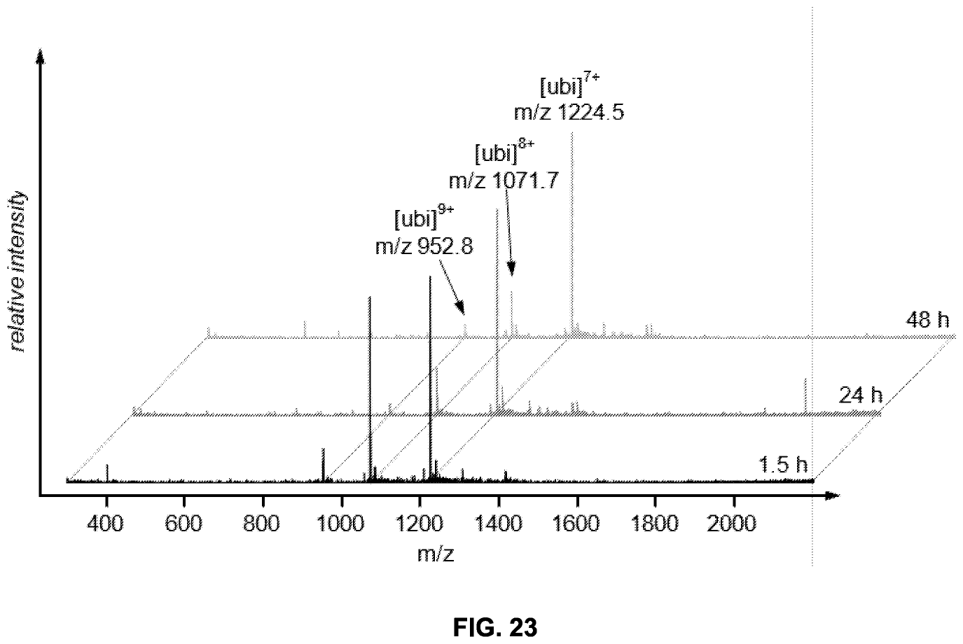

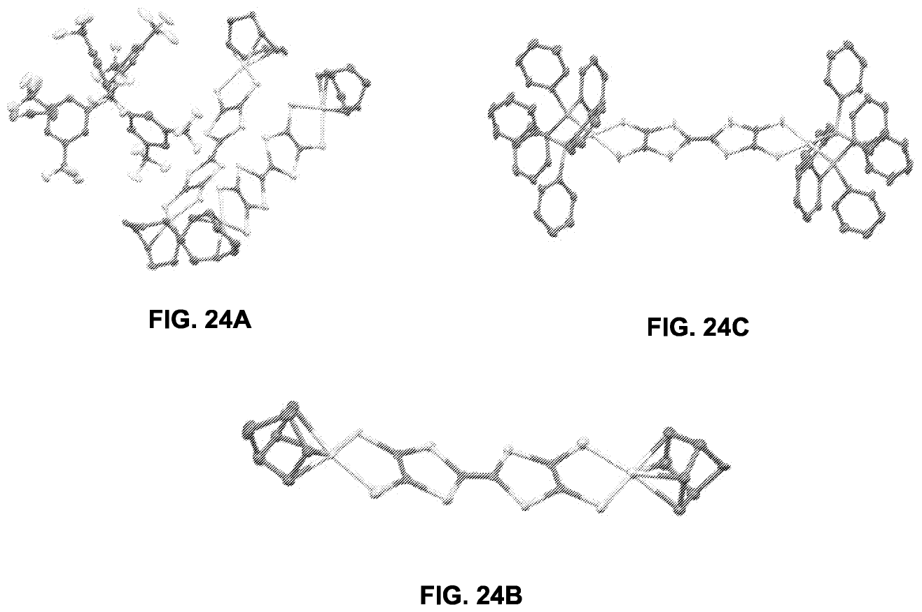

Furthermore, preliminary proteomics studies show that compound 4 is nontoxic and remains intact after exposure to proteins and small nucleophiles (FIGS. 21-23). Compound 4 was also crystallized out of 1:2 MeCN:water over a period of 6 months, demonstrating remarkable stability to water and air over a long period of time (FIG. 24A).

### Example 13

### Synthesis of Compounds 5-9

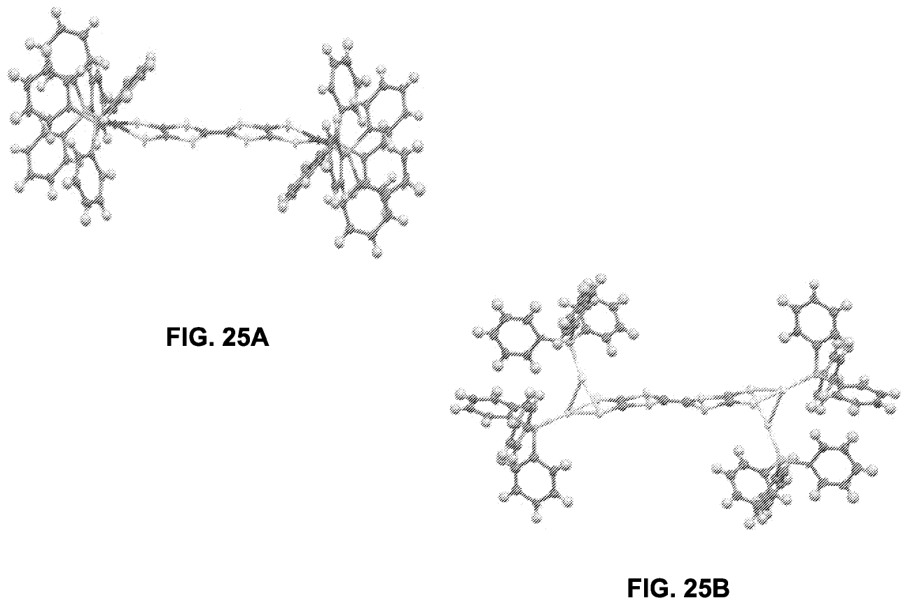

Five other TTFtt complexes were synthesized (four dications, and one neutral TTFtt complex) to further demonstrate the tunability of this scaffold. Compound 5 is the dication PtNBDTTFtt, where NBD is 2,5-norbornadiene (FIG. 24B). Compound 6 is the dication Pt(en)TTFtt, where en is ethylenediamine. Compound 7 is the dication PtPPh₃TTFtt, where PPh₃ is triphenylphosphine, and each platinum center is capped by two triphenylphosphines (FIG. 24C). Compound 8 is PdPPh₃TTFtt, which is analogous to Compound 7, but is capped by two Pd rather than two Pt (FIG. 25A). All the compounds 5-8 are synthesized according to procedures described herein. Compound 9 is Au₄PPh₃TTFtt and is a neutral TTFtt analog, but it is included as it demonstrates that four metals can be bound to one TTFtt moiety (FIG. 25B).

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

1. A method of imaging a target comprising:
(a) administering a detectably effective amount of a tetrathiafulvalene-2,3,6,7 tetrathiolate bridged bimetallic complex to a target in vivo or in vitro;
(b) illuminating the target with a light source emitting light of at least one wavelength or wavelength band causing the tetrathiafulvalene-2,3,6,7 tetrathiolate bridged bimetallic complex to luminesce; and
(c) detecting luminescence with an image detector.

2. The method of claim 1, wherein the tetrathiafulvalene-2,3,6,7 tetrathiolate bridged bimetallic complex is a compound of Formula (I): 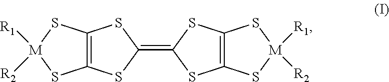
wherein
M is a metal; and
R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring,
and salts thereof.

3. The method of claim 2, wherein the metal is a homometallic, heterometallic or multimetallic complex.

4. The method of claim 3, wherein the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

5. The method of claim 3, wherein the metal is a lanthanide or an actinide.

6. The method of claim 2, wherein the tetrathiafulvalene-2,3,6,7 tetrathiolate bridged bimetallic complex comprises an oxidation product of the compound of Formula (I), wherein the oxidation product comprises a dication diradical, dication or monocation radical of the compound, and an optional counterion.

7. The method of claim 2, wherein the tetrathiafulvalene-2,3,6,7 tetrathiolate bridged bimetallic complex comprises:
(i) an oxidation product of the compound of Formula (I), wherein M is selected from the group consisting of Pt and Pd; and
R₁ and R₂ are independently selected from the group consisting of: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring; and wherein the oxidation product comprises a dication diradical, dication or monocation radical of the compound, and
(ii) at least one anion selected from the group consisting of borates, phosphates, triflates and antimonates, and wherein the at least one anion is optionally covalently bound to at least one capping ligand.

8. The method of claim 1, wherein the tetrathiafulvalene-2,3,6,7 tetrathiolate bridged bimetallic complex is a compound selected from the group consisting of: [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂, [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, and [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

9. The method of claim 1, wherein the target is an organ, a tissue, a muscle, a ligament, a tumor, a surgical site, a sub-cutaneous site, a cell, a neuron, lymphocyte or blood.

10-19. (canceled)

20. A molecular qubit comprising:
a tetrathiafulvalene-2,3,6,7 tetrathiolate bridged bimetallic complex.

21. The molecular qubit of claim 20, wherein the tetrathiafulvalene-2,3,6,7 tetrathiolate bridged bimetallic complex is a compound of Formula (I); 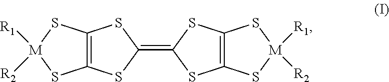
wherein
M is a metal; and
R₁ and R₂ are each independently optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and
wherein R₁ and R₂ taken together may form an optionally substituted ring, and salts thereof.

22. The molecular qubit of claim 21, wherein the metal is a homometallic, heterometallic or multimetallic complex.

23. The molecular qubit of claim 22, wherein the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

24. The molecular qubit of claim 22, wherein the metal is a lanthanide or an actinide.

25. The molecular qubit of claim 21, wherein the tetrathiafulvalene-2,3,6,7 tetrathiolate bridged bimetallic complex comprises an oxidation product of the compound of Formula (I), wherein the oxidation product comprises a dication diradical, dication or monocation radical of the compound, and an optional counterion.

26. The molecular qubit of claim 21, wherein the tetrathiafulvalene-2,3,6,7 tetrathiolate bridged bimetallic complex comprises:
(i) an oxidation product of the compound of Formula (I), wherein M is selected from the group consisting of Pt and Pd; and
R₁ and R₂ are independently selected from the group consisting of: optionally substituted alkylphosphino, optionally substituted alkyl, optionally substituted alkenyl, optionally substituted alkynyl, optionally substituted cyclyl, optionally substituted aralkyl, and optionally substituted aryl, and wherein R₁ and R₂ taken together may form an optionally substituted ring; and wherein the oxidation product comprises a dication diradical, dication or cation of the compound, and
(ii) at least one anion selected from the group consisting of borates, phosphates, triflates and antimonates; and wherein the at least one anion is optionally covalently bound to at least one capping agent.

27. The molecular qubit of claim 20, wherein the tetrathiafulvalene-2,3,6,7 tetrathiolate bridged bimetallic complex is a compound selected from the group consisting of: [(Pd{P(4-trifluoromethylphenyl)₃}₂)₂ tetrathiafulvalene-2,3,6,7 tetrathiolate][tetrakis(3,5-bis(trifluoromethyl)phenyl)borate]₂, [(Pt{bis(diphenylphosphino)ethyl})₂ tetrathiafulvalene-2,3,6,7 tetrathiolate][tetrakis(3,5-bis(trifluoromethyl)phenyl)borate]₂, [(Pt{1,5-cyclooctadiene})₂ tetrathiafulvalene-2,3,6,7 tetrathiolate][tetrakis(3,5-bis(trifluoromethyl)phenyl)borate]₂, and [(Pt{P(4-trifluoromethylbenzyl)₃}₂)₂ tetrathiafulvalene-2,3,6,7 tetrathiolate][tetrakis(3,5-bis(trifluoromethyl)phenyl)borate]₂.

28-35. (canceled)

36. A chromophoric composition comprising:
an oxidation product of a coordination complex of Formula (I): 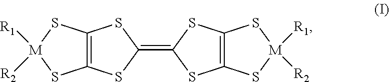
wherein
M is a metal; and
R₁ and R₂ are each independently a therapeutic moiety, optionally substituted aliphatic, optionally substituted heteroaliphatic, optionally substituted heteroalkyl, optionally substituted aromatic, optionally substituted heterocycloalkyl, optionally substituted aryl, or optionally substituted arylalkylene; and wherein R₁ and R₂ taken together may form an optionally substituted ring;
wherein said oxidation product comprises a dication diradical of the coordination complex, and an optional counterion.

37. The chromophoric composition of claim 36, wherein the metal is a homometallic, heterometallic or multimetallic complex.

38. The chromophoric composition of claim 37, wherein the metal is Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Si, Sn, Ge, or Pb.

39. The chromophoric composition of claim 37, wherein the metal is a lanthanide or an actinide.

40. The chromophoric composition of claim 36, wherein at least one of R₁ and R₂ is the therapeutic moiety.

41. The chromophoric composition of claim 40, wherein the therapeutic moiety is
-L-T, wherein L is a linking moiety and T is a biologically active agent.

42. The chromophoric composition of claim 36, wherein R₁ and R₂ are taken together to form a substituted ring, and the therapeutic moiety is a substituent of the substituted ring.

43. The chromophoric composition of claim 41, wherein the linking moiety is selected from the group consisting of: —O—, —SO₂—, —NH—, —C(O)—, —C(O)O—, —OC(O)—, optionally substituted alkyl, optionally substituted alkylene, optionally substituted haloalkylene, optionally substituted hydroxyalkylene, optionally substituted alkylencoxy, optionally substituted heteroalkylene, optionally substituted arylene, optionally substituted aryleneoxy, optionally substituted heterocyclyl and optionally substituted heterocyclyldiyl.

44. The chromophoric composition of claim 41, wherein the biologically active agent is an antineoplastic, antibiotic, corticosteroid, cytotoxic, or immunosuppressive drug.

45. The chromophoric composition of claim 41, wherein the biologically active agent is a protein, antibody, antibody fragment, peptide, aptamer, oligomer, ribonucleic acid or deoxyribonucleic acid.

46. The chromophoric composition of claim 36, further comprises
a pharmaceutically acceptable carrier.

47-75. (canceled)
